(19) United States
(12) Patent Application Publication
(10) Pub. No.: US 2020/0294630 A1
(43) Pub. Date: Sep. 17, 2020

(54) Systems and Methods for Determining Molecular Structures with Molecular-Orbital-Based Features

(71) Applicant: California Institute of Technology, Pasadena, CA (US)

(72) Inventors: Thomas F. Miller, South Pasadena, CA (US); Matthew G. Welborn, Christiansburg, VA (US); Lixue Cheng, Pasadena, CA (US); Tamara Husch, Pasadena, CA (US); Jialin Song, Pasadena, CA (US); Nikola Kovachki, Pasadena, CA (US); Dmitry Burov, Pasadena, CA (US); Ying Shi Teh, Pasadena, CA (US); Anima Anandkumar, Pasadena, CA (US); Feizhi Ding, Pasadena, CA (US); Sebastian Lee, Pasadena, CA (US); Zhuoran Qiao, Pasadena, CA (US); Ali Sahin Lale, Pasadena, CA (US)

(73) Assignee: California Institute of Technology, Pasadena, CA (US)

(21) Appl. No.: 16/817,489

(22) Filed: Mar. 12, 2020

Related U.S. Application Data
(60) Provisional application No. 62/817,344, filed on Mar. 12, 2019.
(60) Provisional application No. 62/821,230, filed on Mar. 20, 2019.
(60) Provisional application No. 62/962,097, filed on Jan. 16, 2020.

(51) Int. Cl. G16C 10/00; G16C 20/70; G16C 20/50; G06N 20/00; G06K 9/62

(52) U.S. Cl. CPC G16C 10/00; G16C 20/70; G06K 9/6232; G06N 20/00; G16C 20/50

(57) ABSTRACT

Systems and methods for determining molecular structures based on molecular-orbital-based (MOB) features are described. MOB features can be utilized in combination with machine-learning methods to predict accurate properties, such as quantum mechanical energy, of molecular systems.

### CROSS-REFERENCE TO RELATED APPLICATIONS

The current application claims the benefit of priority under 35 U.S.C. § 119(e) to U.S. Provisional Patent Application No. 62/817,344 entitled “Harvesting, Databasing, And Regressing Molecular-Orbital Based Features for Accelerating Quantum Chemistry” filed Mar. 12, 2019, U.S. Provisional Patent Application No. 62/821,230 entitled “Molecular-Orbital-Based Features for Machine Learning Quantum Chemistry” filed Mar. 20, 2019, U.S. Provisional Patent Application No. 62/962,097 entitled “Molecular and Materials Discovery and Optimization by Machine Learning with the Use of Molecular-Orbital-Based Features” filed Jan. 16, 2020. The disclosures of U.S. Provisional Patent Application Nos. 62/817,344, 62/821,230, and 62/962,097 are hereby incorporated by reference in its entirety for all purposes.

### GOVERNMENT SPONSORED RESEARCH

This invention was made with government support under Grant No. FA9550-17-1-0102 awarded by US Air Force Office of Scientific Research. The government has certain rights in the invention.

### FIELD OF THE INVENTION

The present invention generally relates to systems and methods to design and synthesize molecules based on molecular system properties; and more particularly to systems and methods that utilize molecular-orbital-based features with machine learning quantum chemistry computing to determine the properties of synthesized chemicals.

### BACKGROUND

Molecular simulations can be helpful to the discovery effort of scientific industry, including solid-state materials, polymers, fine chemicals, and pharmaceuticals. Current approaches employ physics-based methods which solve quantum mechanical equations to describe the behavior of atoms and molecules. While powerful, current methods come at extraordinary computational costs (consuming a sizable fraction of the world's supercomputing resources) and human-time costs (with necessary calculations taking months or longer of wall-clock time). Advances in molecular simulation would broaden its applications in the industrial innovation and development process.

### BRIEF SUMMARY

Systems and methods in accordance with various embodiments of the invention enable the design and/or synthesis of molecules based on molecular system properties. In many embodiments, molecules with specific molecular system properties can be synthesized for a wide range of product development processes such as drug discovery and material design. Examples of materials synthesized in accordance with various embodiments of the invention include (but are not limited to): catalysts, enzymes, pharmaceuticals, proteins and antibodies, organic electronics, surface coatings, nanomaterials, solvents and electrolyte materials that can be used in the construction of batteries.

Many embodiments predict molecular system properties based on molecular orbital based features using molecular-orbital-based machine learning (MOB-ML) processes. Examples of molecular system properties in accordance with various embodiments of the invention include (but are not limited to): solubility, binding affinity for molecules, binding affinity for protein, redox potential, pKa, electrical conductivity, ionic conductivity, thermal conductivity, and light emission efficiency.

In many embodiments, MOB-ML processes can allow for at least 1000-fold speed-ups in computational and wall-clock times over existing physics-based quantum mechanical methods. In several embodiments, the processes allow for at least 100-fold increases in human efficiency. By deploying MOB-ML at scale with cloud resources, the timescale for turnaround can be reduced from days to seconds. MOB-ML in accordance with several embodiments of the invention can enable at least 10-fold prediction accuracy improvements. Some other embodiments implement the software packages, de-risk computational predictions, reduce down-stream experimental and production costs, and accelerate time-to-market.

One embodiment of the invention includes: obtaining a set of molecular orbitals for a molecular system using a computer system; generating a set of molecular-orbital-based features based upon the set of molecular orbitals of the molecular system using the computer system; determining at least one molecular system property based on the set of features using a molecular-orbital-based machine learning (MOB-ML) model implemented on the computer system; and when the determined at least one molecular system property satisfies at least one criterion by the computer system, synthesizing the molecular system.

In a further embodiment, the set of molecular-orbital-based features comprises an attributed graph representation of molecular-orbital-based features.

In another embodiment, the molecular system is one of a plurality of candidate molecular systems. In addition, determining when the determined at least one molecular system property satisfies at least one criterion further includes: generating a set of molecular-orbital-based features based upon sets of molecular orbitals for each of the candidate molecular systems; determining at least one molecular system property for each of the candidate molecular systems based on the set of molecular-orbital-based features of each of the candidate molecular systems using the MOB-ML model; screening the candidate molecular systems based upon the at least one molecular system property determined for each of the candidate molecular systems; and identifying the molecular system based upon the screening.

A still further embodiment also includes training the MOB-ML model to learn relationships between sets of molecular-orbital-based features and molecular system properties using a training dataset describing a plurality of molecular systems and their molecular system properties.

In still another embodiment, training the MOB-ML model to learn relationships between sets of molecular-orbital-based features and molecular system properties further includes: obtaining a set of molecular orbitals for each molecular system in the training dataset of molecular systems by determining occupied molecular orbitals; and obtaining a set of molecular-orbital-based features based upon at least the occupied molecular orbitals.

In a yet further embodiment, a localization process is used to determine occupied molecular orbitals.

In yet another embodiment, obtaining the set of molecular-orbital-based features further comprises performing a dimensionality reduction process on an initial set of features.

In a further embodiment again, the dimensionality reduction process is selected from the group consisting of selecting the molecular-orbital-based features from the initial set of features, and applying a transformation process to the initial set of features to obtain the molecular-orbital-based features.

In another embodiment again, the transformation process is selected from the group consisting of subspace embedding and autoencoding.

In a further additional embodiment, training the MOB-ML model comprises at least one process selected from the group consisting of regression clustering, regression, and classification.

In another additional embodiment, training the MOB-ML model comprises at least regression process selected from the group consisting of Gaussian Process Regression, Neural Network Regression, Linear Regression, and Kernel Ridge Regression with feature selection based on Random Forest Regression, Kernel Ridge Regression without feature selection based on Random Forest Regression, and Kernel Ridge Regression with feature transformation based on Principle Component Analysis.

In a still yet further embodiment, the molecular system comprises at least one of atoms, molecular bonds, and molecules formed by atoms and molecular bonds.

In still yet another embodiment, the set of features includes molecular-orbital-based (MOB) features comprising an energy operator.

In a still further embodiment again, the molecular-orbital-based features further comprise at least one feature selected from the group consisting of: elements from a Fock matrix, elements from a Coulomb matrix, and elements from an exchange matrix.

In still another embodiment again, the at least one molecular system property comprises at least one property selected from the group consisting of quantum correlation energy, force, vibrational frequency, dipole moment, response property, excited state energy and force, and spectrum.

In a still further additional embodiment, the synthesized molecular system comprises at least one molecule selected from the group consisting of a catalyst, an enzyme, a pharmaceutical, a protein, an antibody, a surface coating, a nanomaterial, a semiconductor, a solvent for a battery, and an electrolyte for a battery.

Still another additional embodiment includes: obtaining set of molecular orbitals fora plurality of candidate molecular systems using a computer system; generating a set of molecular-orbital-based features for each candidate molecular system based upon sets of molecular orbitals for each of the candidate molecular systems using the computer system; determining at least one molecular system property for each of the candidate molecular systems based on the set of molecular-orbital-based features of each of the candidate molecular systems using a molecular-orbital-based machine learning (MOB-ML) model implemented on the computer system; screening the candidate molecular systems to identify at least one molecular system possessing at least one molecular system property that satisfies at least one criterion based upon the at least one molecular system property determined for each of the candidate molecular systems using the computer system; and generating a report describing the at least one molecular system identified during the screening of the candidate molecular systems using the computer system.

A yet further embodiment again includes: searching for a set of molecular-orbital-based features having at least one molecular system property predicted by a molecular-orbital-based machine learning (MOB-ML) model that satisfies at least one criterion using a computer system, where the MOB-ML model is trained to receive a set of molecular-orbital-based features of a molecular system and output an estimate of at least one molecular system property; mapping a located set of molecular-orbital-based features to an identified molecular system based upon a feature-to-structure map using the computer system, where the feature-to-structure map is trained to map a set of molecular-orbital-based features to a corresponding molecule structure; and generating a report describing the identified molecular system using the computer system.

Yet another embodiment again also includes screening the identified molecular system based upon at least one molecular system criterion.

Another further embodiment includes: searching for a set of molecular-orbital-based features having at least one molecular system property predicted by a molecular-orbital-based machine learning (MOB-ML) model that satisfies at least one criterion using a computer system, where the MOB-ML model is trained to receive a set of features of a molecular system and output an estimate of at least one molecular system property; mapping a located set of molecular-orbital-based features to an identified molecular system using a feature-to-structure map using the computer system, where the feature-to-structure map is trained to map a set of molecular-orbital-based features to a corresponding molecule structure; screening the identified molecular system based upon at least one screening criterion using the computer system; and when the identified molecular system satisfies the at least one screening criterion, synthesizing the identified molecular system.

In yet another further embodiment, searching for a set of molecular-orbital-based features having at least one molecular system property predicted by the MOB-ML model that satisfies at least one criterion further comprises using at least one generative model to generate candidate sets of features.

In still another further embodiment, the generative model is selected from the group consisting of a variational autoencoder (VAE) and a Generative Adversarial Network (GAN).

Another further embodiment again includes: obtaining a training dataset of molecular systems and their molecular system properties using a computer system; generating a set of molecular-orbital-based features for each molecular system in the training dataset based upon a set of molecular orbitals for each of the candidate molecular systems using the computer system; training a ML model to learn relationships between the set of molecular-orbital-based features of each molecular system in the training dataset and the molecular system properties of each of the molecular systems in the training dataset using the computer system; and utilizing the MOB-ML model to predict at least one molecular system property for a specific molecular system based upon a set of molecular-orbital-based features generated for the specific molecular system based upon a set of molecular orbitals for the specific molecular system.

In another further additional embodiment, obtaining a training dataset of molecular systems and their molecular system properties further includes: generating a set of molecular-orbital-based features for the specific molecular system based upon a set of molecular orbitals for the specific molecular system using the computer system; retrieving molecular-orbital-based features from a database based upon proximity between a retrieved molecular-orbital-based feature and a molecular-orbital-based feature from the set of molecular-orbital-based features for the specific molecular system; and forming the training dataset using the retrieved molecular systems.

In still yet another further embodiment, training the MOB-ML model to learn relationships between the sets of molecular-orbital-based features of each molecular system in the training dataset and the molecular system properties of each of the molecular systems in the training dataset further comprises utilizing a transfer learning process to train an MOB-ML model previously trained to determine the relationship between a molecular-orbital-based features of a molecular system and a different set of molecular system properties.

In still another further embodiment again, training the MOB-ML model to learn relationships between the sets of molecular-orbital-based features of each molecular system in the training dataset and the molecular system properties of each of the molecular systems in the training dataset further comprises utilizing an online learning process to update a previously trained MOB-ML model.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure

### BRIEF DESCRIPTION OF THE DRAWINGS

The description will be more fully understood with reference to the following figures, which are presented as exemplary embodiments of the invention and should not be construed as a complete recitation of the scope of the invention. It should be noted that the patent or application file contains at least one drawing executed in color. Copies of this patent or patent application publication with color drawing(s) will be provided by the Office upon request and payment of the necessary fee.

FIG. 1 illustrates a molecular-orbital-based machine learning process in accordance with an embodiment of the invention.

FIG. 2 illustrates a user interface for software that enables determination of molecular structures in accordance with an embodiment of the invention.

FIG. 3 illustrates diagonal pair correlation energy for a localized σ-bond in water, ammonia, methane, and hydrogen fluoride molecules determined in accordance with an embodiment of the invention.

FIG. 4 conceptually illustrates a database of orbital pairs in accordance with an embodiment of the invention.

FIG. 5 illustrates an MOB-ML process for harvesting molecular-orbital-based features in accordance with an embodiment of the invention.

FIG. 6 illustrates an MOB-ML process to determine molecular system properties incorporating machine learning regression in accordance with an embodiment of the invention.

FIG. 7 illustrates the Greedy algorithm used in regression clustering of an MOB-ML process with an embodiment of the invention.

FIG. 8 illustrates an MOB-ML clustering, regression, and classification process in accordance with an embodiment of the invention.

FIG. 9A illustrates a process for selecting a candidate molecular system to synthesize using an MOB-ML model in accordance with an embodiment of the invention.

FIG. 9B illustrates a process for identifying a molecular system to synthesize using an inverse molecule design process based upon an ML model in accordance with an embodiment of the invention.

FIG. 9C illustrates an MOB-ML process for generating training data relevant to a specific molecular system for the purposes of training an MOB-ML model for use in the estimation of at least one chemical property of the specific molecular system in accordance with an embodiment of the invention.

FIG. 10 illustrates a process for querying a database generated using MOB-ML in accordance with an embodiment of the invention.

FIG. 11 illustrates feature sets and number of features for the diagonal (fᵢ) and off-diagonal (fᵢⱼ) pairs used in MOB-ML training process in accordance with an embodiment of the invention.

FIGS. 12A-12F illustrate MOB-ML predictions of MP2 and CCSD correlation energies and the total correlation energies for a water molecule in accordance with an embodiment of the invention.

FIG. 13 illustrates decomposition of MOB-ML predictions of CCSD correlation energies for a collection of small molecules, with number of training and testing geometries in accordance with an embodiment of the invention.

FIG. 14 illustrates decomposition of MOB-ML predictions of MP2 correlation energies for a collection of small molecules, with number of training and testing geometries in accordance with an embodiment of the invention.

FIG. 15 illustrates MOB-ML predictions of correlation energy of different water molecule geometries at MP2, CCSD, CCSD(T) levels of the post-Hartree-Fock theory, where the MOB-ML process are trained on the water molecule in accordance with an embodiment of the invention.

FIGS. 16A-16C illustrate MOB-ML predictions of CCSD correlation energies for a water tetramer in FIG. 16A, a water pentamer in FIG. 16B, and a water hexamer in FIG. 16C, where the predictions are made with an MOB-ML process trained using a water monomer and water dimer in accordance with an embodiment of the invention.

FIGS. 17A-17C illustrate MOB-ML predictions of MP2 correlation energies for a water tetramer in FIG. 17A, a water pentamer in FIG. 17B, and a water hexamer in FIG. 17C, where the predictions are made with an MOB-ML process trained using a water monomer and a water dimer in accordance with an embodiment of the invention.

FIGS. 18A and 18B illustrate MOB-ML predictions of CCSD correlation energies for butane and isobutane made using an MOB-ML process trained using methane and ethane in FIG. 18A, and made using an MOB-ML process trained from methane, ethane, and propane in FIG. 18B in accordance with several embodiments of the invention.

FIGS. 19A and 19B illustrate MOB-ML predictions of MP2 correlation energies for butane and isobutane made using an MOB-ML process trained from methane and ethane in FIG. 19A, and made using an MOB-ML process trained using methane, ethane, and propane in FIG. 19B in accordance with several embodiments of the invention.

FIG. 20 illustrates MOB-ML predictions of CCSD correlation energies for n-butane and isobutane made using an MOB-ML process trained from ethane and propane in accordance with an embodiment of the invention.

FIGS. 21A and 21B illustrate MOB-ML predictions of CCSD correlation energies for methane, water, and formic acid in FIG. 21A, and for methanol in FIG. 21B, where the predictions are made using an MOB-ML process trained from methane, water, and formic acid in accordance with an embodiment of the invention.

FIGS. 22A and 22B illustrate MOB-ML predictions of MP2 correlation energies for methane, water, and formic acid in FIG. 22A, and for methanol in FIG. 22B, where the predictions are made using an MOB-ML process trained from methane, water, and formic acid in accordance with an embodiment of the invention.

FIG. 23 illustrates MOB-ML predictions of CCSD correlation energies for ammonia, methane, and hydrogen fluoride made using an MOB-ML process trained from water in accordance with an embodiment of the invention.

FIG. 24 illustrates MOB-ML predictions of MP2 correlation energies for ammonia, methane, and hydrogen fluoride made using an MOB-ML process trained from water in accordance with an embodiment of the invention.

FIG. 25 illustrates the number of features selected as a function of the number randomly chosen training molecules for the QM7b-T training dataset at the CCSD(T)/cc-pVDZ level in accordance with an embodiment of the invention.

FIG. 26A illustrates an MOB-ML process learning curve trained on a QM7b-T dataset and applied to a QM7b-T dataset at the MP2/cc-pVTZ and CCSD(T)/cc-pVDZ level in accordance with an embodiment of the invention.

FIG. 26B illustrates an MOB-ML process learning curve trained on a QM7b-T dataset and applied to a GDB-13-T dataset at the MP2/cc-pVTZ level in terms of mean absolute error per heavy atom in accordance with an embodiment of the invention.

FIG. 26C illustrates an MOB-ML process learning curve trained on a QM7b-T dataset and applied to a GDB-13-T dataset in terms of mean absolute error per heavy atom on a logarithmic scale in accordance with an embodiment of the invention.

FIG. 27A illustrates the overlap of clusters obtained via regression clustering for the training set molecules from QM7b-T in accordance with an embodiment of the invention.

FIG. 27B illustrates classification of the data points for the test molecules from QM7b-T using a random forest classifier in accordance with an embodiment of the invention.

FIG. 28 illustrates the analysis of clustering and classification in terms of chemical intuition in accordance with an embodiment of the invention.

FIG. 29 illustrates the sensitivity of MOB-ML predictions for the diagonal and off-diagonal contributions to the correlation energy for the QM7b-T set of training molecules in accordance with an embodiment of the invention.

FIG. 30A illustrates learning curves of an MOB-ML process applied to MP2/cc-pVTZ correlation energies in accordance with an embodiment of the invention.

FIG. 30B illustrates learning curves of an MOB-ML process applied to CCSD(T)/cc-pVDZ correlation energies in accordance with an embodiment of the invention.

FIG. 31 illustrates training costs and transferability of an MOB-ML process with clustering and without clustering applied to correlation energies at the MP2/cc-pVTZ level in accordance with an embodiment of the invention.

FIG. 32A illustrates learning curves of an MOB-ML process applied to MP2/cc-pVTZ correlation energies with and without clustering versus FCHL18 process and FCHL19 process for QM7b-T datasets in accordance with an embodiment of the invention.

FIG. 32B illustrates learning curves of MOB-ML process applied to MP2/cc-pVTZ correlation energies with and without clustering versus an FCHL18 process, and an FCHL19 process for GDB-13-T using the models obtained during the processes illustrated in FIG. 32A in accordance with an embodiment of the invention.

FIG. 33A illustrates the effect of cluster-size capping on MOB-ML prediction accuracy versus the number of training molecules in accordance with an embodiment of the invention.

FIG. 33B illustrates the effect of cluster-size capping on MOB-ML prediction accuracy versus parallelized training time in accordance with an embodiment of the invention.

### DETAILED DESCRIPTION

Turning now to the drawings, systems and methods for synthesizing molecules with specific molecular system properties are described. A molecular system can be atoms, molecular bonds, and/or the resulting molecules formed by the atoms and molecular bonds. Many embodiments implement a molecular-orbital-based machine learning (MOB-ML) process to determine properties of a molecular system. In a number of embodiments, an MOB-ML generative model is utilized to perform generative design of molecular systems having particular desirable properties that can then be synthesized.

In several embodiments, specific molecular system properties are utilized as inputs of an MOB-ML process. In many embodiments, the input properties of the molecular system are a set of features based on molecular orbitals. In some embodiments, the MOB features can be energy operators of the quantum system of the molecular systems. In a number of embodiments, the input MOB features include (but are not limited to): elements of a Fock matrix, elements of a Coulomb matrix, and/or elements of an exchange matrix. As can readily be appreciated, the specific MOB features used to describe a molecular system in accordance with various embodiments of the invention are largely only limited by the requirements of specific applications.

In many embodiments, the MOB-ML processes utilize models that are trained using input datasets. Many embodiments predict certain properties of a molecular system as outputs based on relationships between the input MOB features and the properties that are learned during the training of the MOB-ML model. In some embodiments, the output properties can include (but are not limited to): (1) computable properties of molecules such as electronic energies, correlation energies, forces, vibrational frequencies, dipole moments, response properties, excited state energies and forces, and/or spectra; and (2) experimentally measurable properties of molecules such as activity coefficients, pKa, pH, partition coefficients, vapor pressures, melting, boiling, and flash points, solvation free energies, electrical conductivity, viscosity, toxicity, ADME properties, and protein binding affinities. In several embodiments, a molecular system is selected based upon the predicted property for the molecular system output by the MOB-ML model based upon the input MOB features of the molecular system. In a number of embodiments, the MOB-ML model can be used to perform generative design in which a search is performed within feature space to identify at least one set of MOB features that provide a desired molecular system property. In several embodiments, MOB features can be mapped to molecular structures using a feature-to-structure map that can be derived from a training data set using a machine learning process. The molecular system(s) corresponding to the identified set(s) of MOB features can then be further analyzed to determine the molecular system(s) most suited to a particular application. As can readily be appreciated, systems and methods in accordance with various embodiments of the invention can utilize any of a variety of input MOB features of a molecular system to predict any of a variety of different properties of a corresponding molecular system as appropriate to the requirements of specific applications.

In several embodiments, the molecular systems predicted by the output properties can be in the same molecular family as the input molecular systems. In many embodiments, the molecular systems predicted by the output properties can be in a different molecular family as the input molecular systems. Examples of different molecular families can include (but are not limited to): molecular compositions, molecular geometries, and/or bonding environments. Sets of input MOB features in many embodiments have no explicit dependence on atom types, thus MOB-ML processes can enhance chemical transferability of the training results. In a number of embodiments, the MOB-ML processes are implemented as software applications.

In many embodiments, more complex models of molecular systems can be utilized including (but not limited to) graph organized MOB representations of molecular systems, as an alternative to the current matrix organized MOB representations. In a number of embodiments, quantum chemical information can be represented as an attributed graph G(V,E, X, X^{e}). In certain embodiments, the node features of the attributed graph correspond to diagonal MOB features (Xᵤ=[FᵤᵤJᵤᵤ,Kᵤᵤ]) and the edge features correspond to off-diagonal MOB features (X^{e}ᵤ=[FᵤᵥJᵤᵥ,Kᵤᵥ]). Graph based representations of molecular systems can enable multi-task learning. As can readily be appreciated, appropriately constructed graph representations can provide the benefit of permutation invariance and size extensivity. In many embodiments, a generalized message passing neural network (MPNN) can be utilized to perform the machine learning task from the graph-based representations to a diverse set of chemical properties. In a number of embodiments, MOB-ML processes can utilize graph representations of molecular systems to form general chemical property classification.

Previous work in quantum chemistry has focused on predicting electronic energies or densities based on atom- or geometry-specific features, such as atom-types and bonding connectivities. (See, e.g., Smith, J., et al., Chem. Sci., 2017, 8, 3192-3203; McGibbon, R. T., et al., J. Chem. Phys., 2017, 147, 161725; the disclosures of which are incorporated herein by reference). Such approaches can yield good accuracy with computational cost that is comparable to classical force fields. However, a disadvantage of the approach is that building a machine learning (ML) model to describe a diverse set of elements and chemistries can require training with respect to a number of features that grows quickly with the number of atom- and/or bond-types, and can also require vast amounts of reference data for the selection and training of those features. These issues have hindered the degree of chemical transferability of existing ML models for electronic structure. In addition, across chemical sciences and industries, computation can be hindered by the interplay between prediction accuracy and computational efficiency.

MOB-ML processes in accordance with several embodiments of the invention can improve efficiency and accuracy in quantum simulation. In a number of embodiments, the output properties generated from MOB-ML processes are transferable and thus can be used to determine molecules of different molecular systems. In some embodiments, MOB-ML processes possess transferability across molecular geometries. Several embodiments implement MOB-ML processes with transferability within a molecular family. Some embodiments implement MOB-ML processes providing transferability across bonding environments. Certain embodiments implement MOB-ML processes providing transferability across chemical elements.

Many embodiments implement chemical transferability of MOB-ML processes across molecular systems and so are capable of identifying molecules with a broad range of properties. Molecules with specific molecular system properties can be synthesized using processes in accordance with various embodiments of the invention for a wide range of product development processes such as drug discovery and material design. Examples of such embodiments include (but are not limited to): catalyst design, enzyme reactions and drug design, protein and antibody design, surface coatings, nanomaterials, solvent and electrolyte materials for batteries.

In several embodiments, the transferability of MOB-ML models is leveraged in transfer learning processes that utilize pre-trained energy based models that are transferred to general molecular properties. In a number of embodiments, the transfer learning process can include (but is not limited to) a Gaussian Process kernel transfer and/or a Neural Network based transfer learning process. Furthermore, as increasing amounts of quantum simulation data are generated, MOB-ML processes in accordance with many embodiments of the invention can actively update underlying MOB-ML models based upon new data without requiring retraining using the original training data corpus.

Systems and methods for synthesizing molecules with specific molecular system properties and molecular-orbital-based machine learning (MOB-ML) processes that can be utilized in the design and/synthesis of molecules in accordance with various embodiments of the invention are discussed further below.

### Molecular-Orbital-Based Machine Learning Process

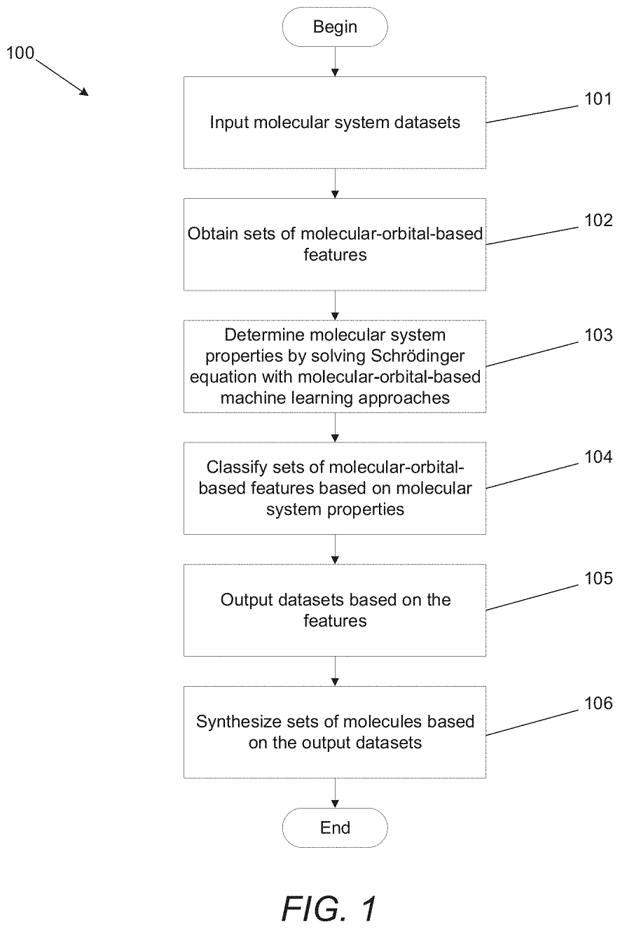

Many embodiments utilize accurate and transferable MOB-ML processes to predict properties including (but not limited to) correlated wavefunction energies based on input features using computations including (but not limited to) a self-consistent field calculation. A method for synthesizing molecules using a MOB-ML process in accordance with an embodiment of the invention is illustrated in FIG. 1. The process 100 can begin by obtaining a molecular system dataset (101). Some embodiments include input datasets that include molecules with the same elements. In a number of embodiments, input datasets can include molecules with different types of molecular bonds. In several embodiments, input datasets can include molecules with different geometries. Some embodiments include input datasets that include different compositions of the same elements. In many embodiments, datasets can include different molecules and elements. As can readily be appreciated, any of a variety of input datasets can be utilized as appropriate to the requirements of specific applications in accordance with various embodiments of the invention.

Sets of MOB features for the input datasets can be obtained based on molecular orbitals (102). In some embodiments, the MOB features can include (but are not limited to) energy operators of the molecular systems. In several embodiments, input MOB features can include (but are not limited to): elements of a Fock matrix, elements of a Coulomb matrix, and/or elements of an exchange matrix. As can readily be appreciated, any of a variety of input MOB features can be utilized as appropriate to the requirements of specific applications.

In certain embodiments, quantum chemistry calculations are performed using MOB-ML processes (103). In a number of embodiments, the computations can be performed on a local computing device. In several embodiments, the calculations are performed on a remote server system. MOB-ML processes can be trained with MOB features of the input datasets.

During a training process (not shown) MOB-ML processes can learn relationships between MOB features and properties of molecular systems using a training dataset. In some embodiments, the training datasets can be subsets randomly selected from input datasets. Examples of molecular datasets in such embodiments can include (but are not limited to): QM7b, QM7b-T, GDB-13, and GDB-13-T. In several embodiments, the training datasets can be sets of molecules from the same or different molecular systems. As can readily be appreciated, any of a variety of training datasets can be utilized as appropriate to the requirements of specific applications in accordance with various embodiments of the invention.

The MOB-ML processes can utilize a trained model that describes relationships between MOB features and properties of molecular systems to perform a ranking and/or categorization (104) of at least the molecules in the input dataset. In many embodiments, the MOB-ML processes can also identify novel molecules and/or molecules that are not in the input dataset based upon regions of the feature space that contain molecules that the model predicts will have desirable properties. The various ways in which MOB-ML processes can be utilized to identify molecular systems having desirable properties in accordance with various embodiments of the invention including specific examples are discussed further below.

In many embodiments, the trained MOB-ML processes generate output datasets of molecular system properties (105). The molecular system properties can include (but are not limited to): (1) computable properties of molecules such as electronic energies, correlation energies, forces, vibrational frequencies, dipole moments, response properties, excited state energies and forces, and/or spectra; and (2) experimentally measurable properties of molecules such as activity coefficients, pKa, pH, partition coefficients, vapor pressures, melting, boiling, and flash points, solvation free energies, electrical conductivity, viscosity, toxicity, ADME properties, and protein binding affinities. As can readily be appreciated, the specific features used as molecular system properties are largely only limited by the requirements of specific applications. Based on the output datasets, molecules with sets of desired molecular system properties can be identified and synthesized (106).

While various processes for synthesizing chemicals using MOB-ML processes are described above with reference to FIG. 1, any of a variety of processes that utilize machine learning to estimate the properties of molecular systems can be utilized in the design and/or synthesis of chemicals as appropriate to the requirements of specific applications in accordance with various embodiments of the invention. For example, molecular systems can be synthesized in a process that utilizes a generative MOB-ML process to identify the molecular system as having molecular properties satisfying certain criteria using techniques similar to those discussed below. Processes for designing molecules with desired properties in accordance with various embodiments of the invention are discussed further below.

### Determining Molecular Structures

In many embodiments, MOB-ML processes enable real-time chemical modeling, design, and collaboration. In several embodiments, the MOB-ML processes are implemented in software packages that can execute on a local computer or on a remote server. Additionally, the software packages according to some embodiments, can perform calculations on many possible chemical modifications and return rank-ordered recommendations for the most promising chemical modifications. With parallel computation all of the results can be returned in seconds. In this way, processes similar to the various processes for designing molecular systems described above can be performed and the results used to generate intuitive and interactive graphical user interfaces that enable any of a variety of experimental chemists to utilize MOB-ML in the design and/or synthesis of chemicals.

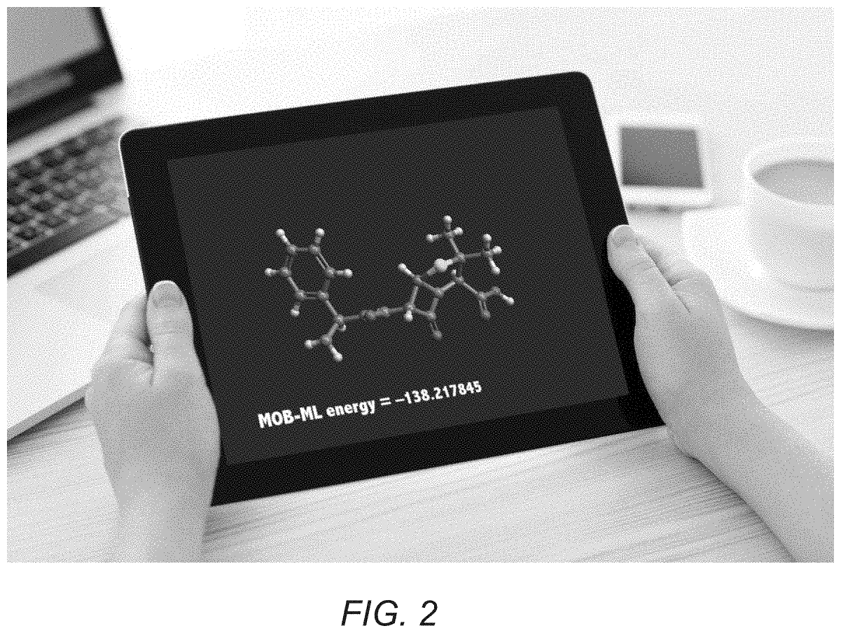

A user interface that can be generated by software using a ML process implemented in accordance with an embodiment of the invention is conceptually illustrated in FIG. 2. In many embodiments, the software can enable any experimental chemist, instead of only expert computational chemists, to identify molecular systems possessing desirable chemical properties. For example, user interfaces can be implemented for the software that can enable the design and synthesis of molecular systems by any of a variety of experimental chemists including (but are not limited to): medicinal chemists, synthetic chemists, material scientists, and/or biochemists.

While various processes for designing molecules using MOB-ML processes are described above with reference to FIG. 2, any of a variety of processes that utilize machine learning to estimate the properties of molecular systems can be utilized in the design and synthesis of chemicals as appropriate to the requirements of specific applications in accordance with various embodiments of the invention. Processes for performing MOB feature generation in accordance with various embodiments of the invention are discussed further below.

### Molecular-Orbital-Based Feature Generation

Dimensionality reduction of the features of molecular systems can be an important part of an MOB-ML process implemented in accordance with an embodiment of the invention. The high dimensionality of the full set of features that can be generated by a molecular system can lead to over-fitting of dimensions that serve little informative value. Many embodiments include a variety of processes that can be utilized to generate features. Many embodiments include a variety of processes that perform a dimensionality reduction of features including (but not limited to) through feature selection and/or feature transformation. Some embodiments select features based on Hartree-Fock molecular orbitals to predict post-Hartree-Fock correlated wavefunction energies. Some embodiments are based on features of orbitals defined in (tight-binding) density functional theory calculations. Several embodiments include elements of a Fock matrix, elements of a Coulomb matrix, and/or elements of an exchange matrix as features. As can readily be appreciated, any of a variety of operations can be evaluated for the molecular orbitas which can be used as input MOB features and any of a variety of input MOB features can be selected as appropriate to the requirements of a specific application. In several embodiments, dimensionality reduction can also be achieved through feature transformation techniques, such as (but not limited to) Principal Component Analysis (PCA), truncated Singular Value Decomposition (SVD), and Neural Networks. Furthermore, in a number of embodiments, MOB features can be utilized to directly train a MOB-ML model without additional dimensionality reduction. As can readily be appreciated, the specific processes for evaluating molecular orbitals, performing dimensionality reduction and/or training MOB-ML models using MOB features are largely dependent upon the requirements of specific applications.

In many embodiments, feature generation includes a canonical ordering of the occupied and virtual molecular orbitals. Several embodiments apply localized molecular orbital (LMOs). In a number of embodiments, MOB features can be obtained from other types of MOs including (but not limited to) canonical and natural orbitals. Some embodiments utilize Boys localization for localization in occupied space and Intrinsic Bonding Orbital (IBO) localization for localization in virtual space. As can readily be appreciated, any of a variety of unitary orbital transformations can be utilized to obtain MOs as appropriate to the requirements of specific applications. In several embodiments, MOB features can be sorted by increasing distance from occupied MOs. As can readily be appreciated, any of a variety of sorting criteria can be utilized as appropriate to the requirements of specific applications. In some embodiments, automatic feature selection can be performed using any of a variety of processes including (but not limited to) random forest regression utilizing a mean decrease of accuracy criterion. As can readily be appreciated, any of a variety of processes can be utilized in the selection of features as appropriate to the requirements of specific applications. Selection and/or sorting is not required, however. A number of embodiments of the invention utilize machine learning models including (but not limited to) Neural Network models that receive MOB features as a direct input and output estimates of molecular properties for the received MOB features as an output. Various ways in which MOB-ML processes can estimate molecular properties from sets of features describing molecular systems in accordance with different embodiments of the invention are discussed further below.

Sets of MOB features in many embodiments have no explicit dependence on atom types, thus MOB-ML processes can enhance chemical transferability of the training results. In several embodiments, the smooth variation and local linearity of pair correlation energies as a function of MOB features of different molecular geometries and different molecules can be beneficial to the transferability of MOB-ML processes.

Many embodiments can predict properties of molecular systems including (but not limited to) post-Hartree-Fock correlated wavefunction energies using MOB features including (but not limited to) the Hartree-Fock (HF) molecular orbitals (MOs). In some embodiments, the starting point for a MOB-ML process involves decomposing the correlation energy into pairwise occupied MO contributions

$${E}_{c} = {\sum }_{ij}^{OCC} {ɛ}_{ij} , \qquad ( 1 )$$

where the pair correlation energy εᵢⱼ can be written as a functional of the full set of MOs, {ϕₚ}, appropriately indexed by i and j

εᵢⱼ=ε[{ϕₚ}^{ij}]. (2)

The functional ε can be considered universal across all chemical systems; for a given level of correlated wavefunction theory, there is a corresponding E that maps the HF MOs to the pair correlation energy, regardless of the molecular composition or geometry. Furthermore, E simultaneously describes the pair correlation energy for all pairs of occupied MOs (i.e., the functional form of E does not depend on i and j). For example, in second-order Møller-Plessett perturbation theory (MP2), the pair correlation energies can be expressed as

$${ɛ}_{i j}^{MP2} = \frac{1}{4} {\sum }_{a b}^{virt} \frac{\vert < ij \vert {\vert ab > \vert}^{2}}{{e}_{a} + {e}_{b} - {e}_{i} - {e}_{j}} \qquad ( 3 )$$

where a and b index virtual MOs, eₚ is the Hartree-Fock orbital energy corresponding to MO ϕ₉, and <ij∥ab> are anti-symmetrized electron repulsion integrals. A corresponding expression for the pair correlation energy can exist for any post-Hartree-Fock method, but it is typically costly to evaluate in closed form.

In MOB-ML, a machine learning model can be constructed for the pair energy functional

εᵢⱼ≈ε^{ML}[fᵢⱼ] (4)

where fᵢⱼ denotes a vector of features associated with MOs i and j. Eq. 4 thus presents the opportunity for the machine learning of a universal density matrix functional for correlated wavefunction energies, which can be evaluated at the cost of the MO calculation.

The features fᵢⱼ can correspond to unique elements of the Fock (F), Coulomb (J), and exchange (K) matrices between ϕᵢ, ϕⱼ and the set of virtual orbitals. Some embodiments include features associated with matrix elements between pairs of occupied orbitals for which one member of the pair differs from ϕᵢ or ϕⱼ (i.e., non-i, j occupied MO pairs). In several embodiments, the feature vector can take the form

fᵢⱼ=(Fᵢᵢ,Fᵢⱼ,Fⱼⱼ,Fᵢ^{O},Fⱼ^{O},Fᵢⱼ^{VV},

Jᵢᵢ,Jᵢⱼ,Jⱼⱼ,Jᵢ^{O},Jᵢ^{V},Jᵢ^{V},Jᵢⱼ^{VV},

Kᵢⱼ,Kᵢ^{O},Kⱼ^{O},Kᵢ^{V},Kⱼ^{V},Kᵢⱼ^{VV}). (6)

where for a given matrix (F, J, or K) the superscript o denotes a row of its occupied-occupied block, the superscript v denotes a row of its occupied-virtual block, and the superscript vv denotes its virtual-virtual block. Redundant elements can be removed, such that the virtual-virtual block is represented by its upper triangle and the diagonal elements of K (which are identical to those of J) are omitted. To increase transferability and accuracy, ϕᵢ and ϕⱼ can be localized molecular orbitals (LMOs) rather than canonical MOs and employ valence virtual LMOs in place of the set of all virtual MOs. In this way, Eq. 4 can be separated to independently machine learn the cases of i=j and i≠j,

$${ɛ}_{i j} \approx \{ \begin{matrix}{ɛ}_{d}^{M L} [ {f}_{i} ] , & if i = j \\ {ɛ}_{o}^{M L} [ {f}_{i j} ] , & if i \neq j\end{matrix} \qquad ( 6 )$$

where fᵢ denotes fᵢᵢ (Eq. 5) with redundant elements removed; by separating the pair energies in this way, the situation where a single ML model is required to distinguish between the cases of i=j and ϕᵢ being nearly degenerate to ϕⱼ is avoided, a distinction which can represent a sharp variation in the function to be learned.

Many embodiments introduce technical refinements to improve training efficiency, for example the accuracy and transferability of the model as a function of the number of training examples.

Some embodiments implement occupied LMO symmetrization. In this way, the feature vector can be pre-processed to specify a canonical ordering of the occupied and virtual LMO pairs. This can reduce permutation of elements in the feature vector, resulting in greater ML training efficiency. Matrix elements Mᵢⱼ (M=F, J, K) associated with ϕᵢ and ϕⱼ can be rotated into gerade and ungerade combinations

$${M}_{ii} ← \frac{1}{2} {M}_{ii} + \frac{1}{2} {M}_{jj} + {M}_{ij} \\ {M}_{jj} ← \frac{1}{2} {M}_{ii} + \frac{1}{2} {M}_{jj} - {M}_{ij} \\ {M}_{ij} ← \frac{1}{2} {M}_{ii} - \frac{1}{2} {M}_{jj} \\ {M}_{ip} ← \frac{1}{\sqrt{2}} {M}_{ip} + \frac{1}{\sqrt{2}} {M}_{jp} \\ {M}_{jp} ← \frac{1}{\sqrt{2}} {M}_{ip} - \frac{1}{\sqrt{2}} {M}_{jp} \qquad ( 7 )$$

with the sign convention that Fᵢⱼ is negative. Here, p indexes any LMO other than i or j, for example an occupied LMO k, such that i≠k≠j, or a valence virtual LMO. As can readily be appreciated, any rotation of pairs of orbitals can be applied as appropriate to the requirements of specific applications.

Several embodiments implement LMO sorting. The LMO pairs can be sorted by increasing distance from occupied orbitals ϕᵢ and ϕⱼ. Sorting in this way can result in features corresponding to LMOs being listed in decreasing order of heuristic importance in such a way that the mapping between LMOs and their associated features is roughly preserved. In some embodiments, the LMO pairs can be sorted by decreasing approximate energy contribution to the correlation energy of the occupied orbitals ϕᵢ and ϕⱼ. As can readily be appreciated, any of a variety of sorting criteria can be utilized as appropriate to the requirements of specific applications.

For purposes of sorting, distance can be defined as

Rₐ^{ij}=∥ [symbol] ϕᵢ|\hat{R}|ϕᵢ [symbol] − [symbol] ϕₐ|\hat{R}|ϕₐ [symbol] ∥+∥ [symbol] ϕⱼ|\hat{R}|ϕⱼ− [symbol] ϕₐ|\hat{R}|ϕₐ [symbol] ∥ (8)

where ϕₐ is a virtual LMO, \hat{R} is the Cartesian position operator, and 11.11 denotes the L2-norm.∥ [symbol] ϕᵢ|\hat{R} |ϕᵢ [symbol] − [symbol] ϕₐ|\hat{R} |ϕₐ [symbol] ∥ represents the Euclidean distance between the centroids of orbital i and orbital a. Distances can be defined based on Coulomb repulsion, which sometimes leads to inconsistent sorting in systems with strongly polarized bonds. The non-i, j occupied LMO pairs can be sorted in the same manner as the virtual LMO pairs. As can readily be appreciated, any of a variety of distance measurements can be utilized as appropriate to the requirements of specific applications in accordance with various embodiments of the invention.

Several embodiments implement orbital localization. In some embodiments, Intrinsic Bonding Orbital (IBO) localization can be used to obtain the occupied LMOs. In a number of embodiments, Boys localization can be used to obtain the occupied LMOs. Particularly for molecules that include triple bonds or multiple lone pairs, Boys localization can provide more consistent localization as a function of small geometry changes than IBO localization; and the chemically unintuitive mixing of σ and π bonds in Boys localization (“banana bonds”) does not present a problem for the MOB-ML process. As can readily be appreciated, any of a variety of unitary orbital transformations can be utilized to obtain MOs as appropriate to the requirements of specific applications in accordance with various embodiments of the invention.

Many embodiments implement dimensionality reduction of MOB features. Prior to training, automatic feature selection and/or transformation can be performed using processes including (but not limited to) random forest regression with the mean decrease of accuracy criterion or permutation importance. Such embodiments implement Gaussian Process Regression (GPR), which has performance that is known to degrade for high-dimensional datasets (in practice 50-100 features). The use of the full feature set with small molecules can lead to overfitting as features become correlated. As can readily be appreciated, any of a variety of sets of MOB features can be utilized to express a feature space of molecular system as appropriate to the requirements of specific MOB-ML and/or molecular synthesis processes in accordance with various embodiments of the invention.

While various processes for MOB feature selection are described above, any variety of processes that utilize quantum theory to select MOB features can be utilized in MOB-ML processes as appropriate to the requirements of specific applications in accordance with various embodiments of the invention. Processes for identifying MOB feature distance metrics in accordance with various embodiments of the invention are discussed further below.

### Chemical Space Structure Discovery

Processes in accordance with various embodiments of the invention can rely upon the use of distance metrics that measure the distance between the MOB features of different molecular systems in feature space. In many embodiments, chemical space structure discovery is further enhanced by utilizing subspace embedding techniques and/or autoencoder techniques to discover the local and global structures of MOB feature space. As is discussed further below, any of a variety of distance measures and/or structure discovery techniques can be utilized as appropriate to the requirements of specific applications in accordance with various embodiments of the invention.

Many embodiments implement MOB features including (but not limited to) a set of distance measures between a pair of molecular orbitals in the space. In this space, a distance can be defined which distinguishes pairs based on their MOB features. Specific implementations can include (but are not limited to): Euclidean distance in the space of MOB features or in a subspace thereof; kernel distance measures such as those employed in Gaussian Process Regression in the space of MOB features or in a subspace thereof, including but not limited to exponential, squared exponential, and Matérn kernels; and measures based on manifold learning in the space of MOB features or in a subspace thereof, including but not limited to diffusion maps, t-stochastic neighbor embedding, and isomap. In embodiments that utilize Gaussian Process Regression and in which kernel distance measures are utilized, the Nystrom method can be utilized to perform sampling of the kernel matrix to enable the Gaussian Process Regression to be performed in a more computationally efficient manner with little or no accuracy loss. Furthermore, the kernels used in Gaussian Process Regression can be extended to functions constructed from MOB feature space using Neural Networks. In certain embodiments, physical intuition can also be incorporated into the construction of the kernel. MOB features can be ordered according to various distance measures in accordance with many embodiments of the invention. As can readily be appreciated, any of a variety of distance metric implementations can be utilized as appropriate to the requirements of specific applications in accordance with various embodiments of the invention.

Appropriately obtained sets of MOB features can provide a faithful and structured representation of chemical space. Exploration and discovery of the local and global structures of an MOB feature space can be facilitated using discovery techniques including (but not limited to) subspace embedding techniques and/or autoencoder techniques. The use of such discovery techniques can enhance MOB-ML process accuracy and/or provide physical insights for chemists to understand trends and similarities across chemical systems. The term subspace embedding is generally used to describe a set of techniques that can simplify the analysis of high dimensional data, which can be especially useful for sparse data. In a number of embodiments, subspace embedding techniques including (but not limited to) Uniform Manifold Approximation and Projection (UMAP), t-Stochastic Neighbor Embedding (t-SNE), and/or Oblivious Subspace Embedding (OSE) are utilized to reduce a high dimensional MOB feature space to a relatively low-dimensional subspace and facilitate chemical space structure discovery in accordance with various embodiments of the invention. Similarly, an autoencoder such as (but not limited to) an autoencoder neural network can be utilized to perform dimensionality reduction by learning a vector subspace embedding for a higher dimensionality MOB feature space. In a number of embodiments, a subspace embedding can be performed that preserves relative distance measurements between sets of MOB features in the higher dimensional MOB feature space to enable exploration of the properties of different sets of MOB features in the lower dimensionality subspace. As can readily be appreciated, the specific subspace embedding process utilized is largely dependent upon the requirements of a given application.

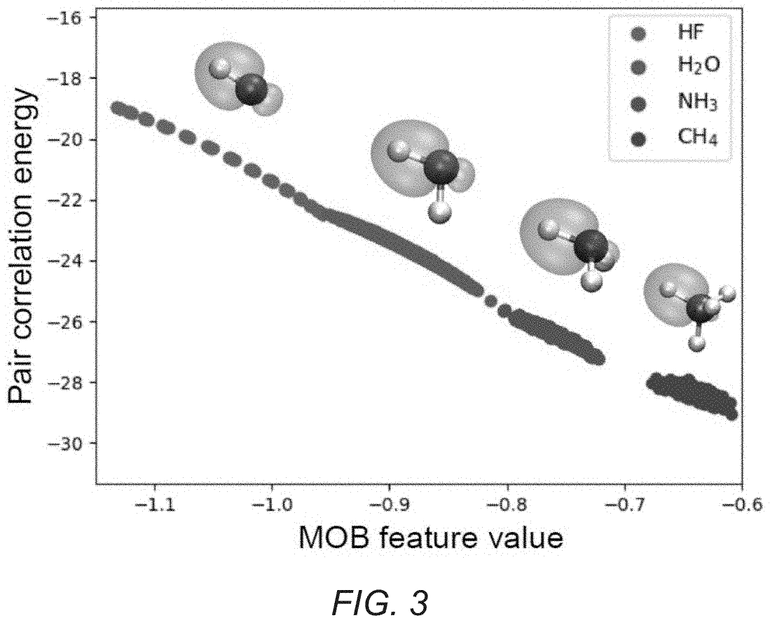

Several embodiments include pair correlation energies as a function of MOB features such that smooth variation and local linearity can be obtained for different molecules with different molecular geometries and hence enhance the transferability of MOB-ML processes. FIG. 3 illustrates σ-bonding orbitals in hydrogen fluoride, water, ammonia, and methane molecules which are encoded in MOB features. The y-axis shows the diagonal contribution to the correlation energy associated with this orbital (εᵢᵢ), computed at the MP2/cc-pvTZ level of theory. The x-axis shows the value of a particular MOB feature, a Fock matrix element for the that localized orbital, Fᵢᵢ. For each molecule, a range of geometries can be sampled from the Boltzmann distribution at 350 K, with each plotted point corresponding to a different sampled geometry. In FIG. 3, the pair correlation energy can vary smoothly and linearly as a function of the MOB feature value. The slope of the linear curve can be consistent across molecules in accordance with an embodiment. Many embodiments include MOB features that can lead to accurate regression of correlation energies using simple machine learning models and linear models. Several embodiments enable the transferability of MOB-ML processes across diverse chemical systems, including systems with elements that do not appear in the training set.

While systems and methods that include various MOB feature distance metrics are described above, any of variety of processes for measuring distance between the MOB features of different molecular systems can be utilized in MOB-ML processes as appropriate to the requirements of specific applications in accordance with various embodiments of the invention. Processes for generating orbital pairs databases in accordance with various embodiments of the invention are discussed further below.

### Generating Databases of Orbital Pairs

Processes in accordance with various embodiments of the invention are capable of generating databases of molecular orbital pairs. As is discussed further below, any of a variety of orbital pair databases can be utilized as appropriate to the requirements of specific applications in accordance with various embodiments of the invention.

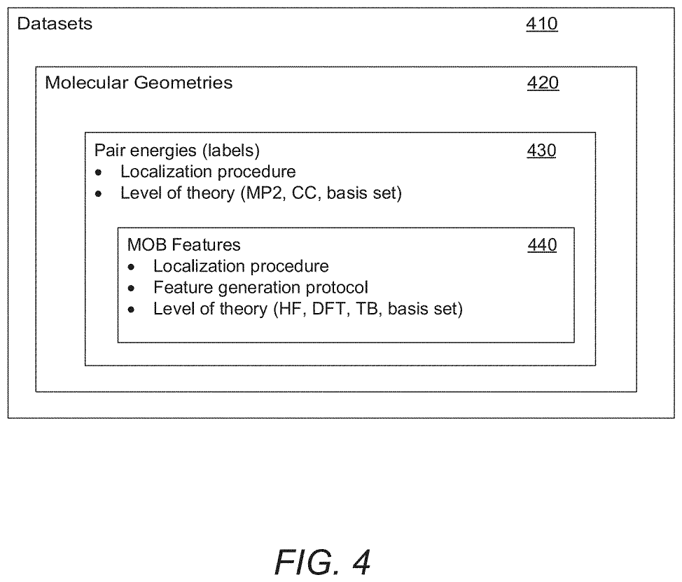

Many embodiments implement MOB-ML processes that store, organize, and classify databases that include (but are not limited to) molecular orbitals which form the basis for the associated MOB feature values. In some embodiments, the MOB feature values can be output from MOB-ML processes, using processes similar to those described above with respect to FIG. 1. In some embodiments, a molecular orbital database is utilized that is organized based on a set of distance measures between a pair of molecular orbitals in the MOB original feature space and/or a subspace and/or latent space of the MOB feature space. FIG. 4 schematically illustrates database structures in accordance with an embodiment of the invention. The databases 410 can contain molecular geometries 420. The molecular geometries can determine (but are not limited to) associated pair energies 430. The associated pair energies can be calculated using processes including (but not limited to) (non)-canonical MP2 theory, and/or coupled cluster theory. The associated pair energies can be utilized to determine input MOB features 440. The MOB features can be determined by (but not limited to) feature generation protocols applying various localization procedures and levels of quantum chemistry theories such as different basis sets from Hartree-Fock (HF), or different basis sets from density function theory (DFT). As can readily be appreciated, the specific features used in the generation of molecular orbital databases are largely only limited by the requirements of specific applications. Furthermore, databases can be generated using more complex representations of quantum chemical information including (but not limited to) attributed graphs. In several embodiments, databases are constructed in which quantum chemical information for molecular systems is described using attributed graphs constructed using molecular-orbital-based features G(V,E, X, X^{e}) with node features Xᵤ=[Fᵤᵤ, Jᵤᵤ, Kᵤᵤ] and edge features X^{e}ᵤ=[Fᵤᵥ, Jᵤᵥ, Kᵤᵥ]. In a number of embodiments, quantum chemical information represented as attributed graphs in this way can be utilized within a variety of MOB-ML processes including (but not limited to) MOB-ML processes that perform multi-task learning to learn associations between the attributed graph structures and chemical properties from a training data set. A benefit of the graph representation is that they can provide permutation invariance and size-extensivity, and be utilized for general chemical property classification or regression utilizing techniques including (but not limited to) a graph neural network incorporating a generalized message-passing mechanism. As can readily be appreciated, quantum chemical information can be represented using any of a variety of techniques and/or structures within databases and the represented information can be utilized in a variety of machine learning and/or generative processes similar to those described herein to facilitate the synthesis of molecular systems having desirable chemical properties as appropriate to the requirements of specific applications. Accordingly, embodiments of the invention should be understood as not being limited to any particular representation of quantum chemical information, but instead by understood as general techniques that are applicable to any representation of quantum chemical information.

The databases 410 can be queried to generate datasets corresponding to particular sets of molecules, molecular geometries, level of theory, or any combination thereof. Various embodiments employ SQL databases such as MySQL or no-SQL databases such as MongoDB distributed across one or more computers. The databases, according to various embodiments, can be queried to find MOB features nearby to a given set of MOB features on the basis of a distance metric measured between a pair of molecular orbitals in the space. Several embodiments enable the databases to be queried to find molecular systems on the basis of the MOB feature values associated with the molecular orbitals associated with those molecular systems. Examples of such embodiments can include (but are not limited to): employing k-d trees in the space of MOB features. As can readily be appreciated, any of a variety of implementations of database indexes and/or to facilitate searching can be utilized as appropriate to the requirements of specific applications in accordance with various embodiments of the invention.

While various processes for generating orbital pairs databases are described above, any variety of orbital pairs databases of different molecular systems can be utilized in MOB-ML processes as appropriate to the requirements of specific applications in accordance with various embodiments of the invention. Processes for harvesting MOB features in accordance with various embodiments of the invention are discussed further below.

### Molecular-Orbital-Based Feature Harvesters

Processes in accordance with various embodiments of the invention rely upon harvesting MOB features from quantum chemistry calculations. As is discussed further below, any of a variety of MOB feature harvesters can be utilized as appropriate to the requirements of specific applications in accordance with various embodiments of the invention.

Many embodiments implement MOB-ML processes to collect and harvest MOB feature values from the output of quantum chemistry calculations. Some embodiments of the MOB feature values collected from the MOB-ML processes can include the MOB feature values based on the distance between a pair of molecular orbitals to the MOB feature values that are stored within a database of molecular orbitals. Some other embodiments of the MOB feature values collected from the MOB-ML processes eliminate the MOB feature values based on the distance between a pair of molecular orbitals to the MOB feature values that are stored within the databases of molecular orbitals.

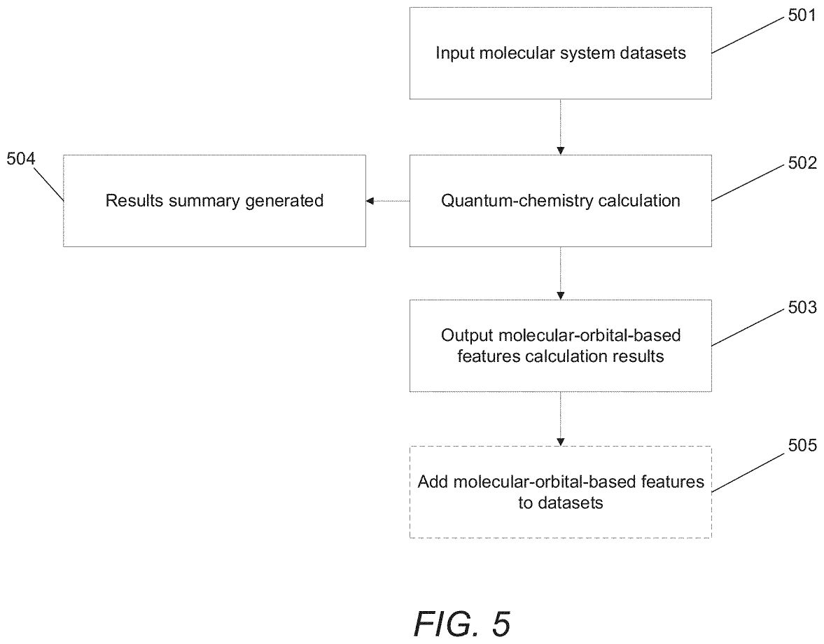

A method for collecting and harvesting MOB features using a MOB-ML process in accordance with an embodiment is illustrated in FIG. 5. Datasets of molecular systems can be generated as input 501. Quantum chemistry calculations can be applied to input datasets 502. The calculation can generate and output corresponding MOB features 503. These features can be stored in a database of molecular orbitals 505. Molecules from the calculation results can also be used for synthesis of such molecules 504.

While various processes for harvesting MOB features are described above, any variety of processes that can collect and harvest MOB features of different molecular systems can be utilized in MOB-ML processes as appropriate to the requirements of specific applications in accordance with various embodiments of the invention. Processes for machine learning regression method in accordance with various embodiments of the invention are discussed further below.

### Machine Learning Regression

Processes in accordance with various embodiments of the invention rely upon machine learning techniques including (but not limited to) machine learning regression. As is discussed further below, any of a variety of machine learning regression methods can be utilized as appropriate to the requirements of specific applications in accordance with various embodiments of the invention.

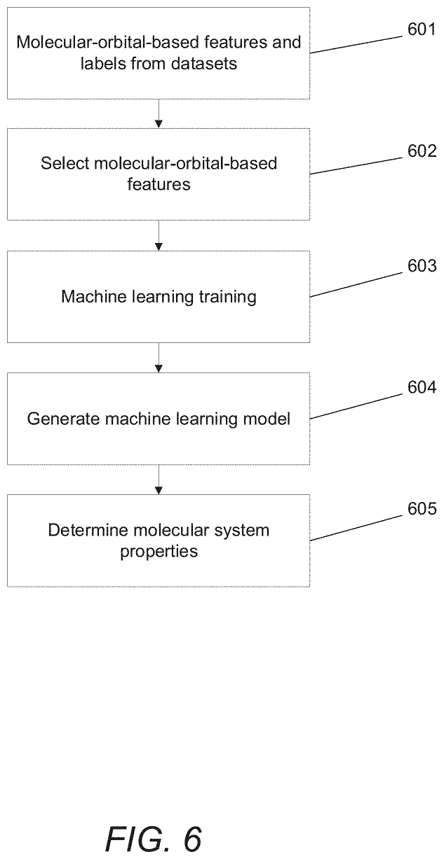

Many embodiments include MOB-ML processes that incorporate molecular orbital databases to determine accurate molecular system properties. Examples of such embodiments are illustrated in FIG. 6. MOB features and labels from accurate reference calculations can be extracted from the molecular orbitals databases 601. Feature selection can be performed 602. A machine learning model can be trained based on the selected MOB features 603. A trained model can be used to predict the labels from these features 604 and/or can be utilized in generative processes. The model may be used to predict accurate molecular system properties including (but not limited to) quantum mechanical energies 605. Such embodiments can include but are not limited to: Gaussian Process Regression, Neural Network Regression, Linear Regression, or Kernel Ridge Regression with or without feature selection based on Random Forest Regression or feature transformation based on Principle Component Analysis. As can readily be appreciated, any of a variety of machine learning regression processes can be utilized as appropriate to the requirements of specific applications in accordance with various embodiments of the invention.

In many embodiments, the molecular system properties that are determined using the MOB-ML process include but are not limited to quantum mechanical energies, forces, vibrational frequencies (hessian), dipole moments, response properties, excited state energies and forces, and spectra. As can readily be appreciated, any of a variety of molecular system properties can be utilized as appropriate to the requirements of specific applications in accordance with various embodiments of the invention. Some embodiments implement the prediction of forces and hessians that can be used to optimize the geometry of the molecular system to a local minimum or saddle point. Several embodiments include that the prediction of forces can be used to run molecular dynamics. Yet some embodiments include the prediction of energies and forces that can be used to perform configurational sampling. The predictions, according to several embodiments, can be made for high-level theories on the basis of MOB feature values that are obtained using a smaller atom-centered basis set. Examples of high-level theories can include (but not limited to) coupled cluster theory using a large atom-centered basis set. As can readily be appreciated, the specific features used as high-level theories are largely only limited to the requirements of specific applications. In some embodiments, the prediction can be made for high-level theories on the basis of MOB feature values that may include data from intermediate-level theories. Examples of high-level theories can include (but are not limited to) coupled cluster theory. As can readily be appreciated, the specific features used as high-level theories are largely only limited to the requirements of specific applications. Examples of intermediate-level theories can include (but are not limited to) MP2 theory. As can readily be appreciated, the specific features used as intermediate-level theories are largely only limited to the requirements of specific applications.

As the amount of quantum simulation data increases, MOB-ML processes in accordance with many embodiments of the invention can utilize online learning techniques to continuously update MOB-ML models without retraining the models using the entirety of the original training data set. In a number of embodiments, variational Gaussian Process formalism can be generalized for minibatched training for efficient online learning within an MOB-ML process. As can readily be appreciated, any of a variety of online ML techniques can be utilized to update previously trained MOB-ML models using additional quantum simulation data as appropriate to the requirements of specific applications in accordance with various embodiments of the invention. In several embodiments, software implementations of MOB-ML models can provide user interfaces that enable a user to efficiently update an existing MOB-ML model using additional sources of quantum simulation data selected by the user including (but not limited) streams of quantum simulation data.

In many instances, limited numbers of quantum simulations and/or experimental data may be available with respect to a particular molecular property. In a number of embodiments, the transferability of MOB-ML models is utilized to perform a transfer learning process that utilizes a MOB-ML trained with respect to a first set of molecular properties as an input to a training process that learns relationships between a set of quantum simulations and/or experimental data and a second set of molecular properties. In several embodiments, pre-trained energy based models can be utilized as inputs to a transfer learning process. In a number of embodiments, a transfer learning process such as (but not limited to) Gaussian Process kernel transfer and/or Neural Network transfer learning processes can be utilized as appropriate to the requirements of specific applications. The well-structured chemical space obtained from MOB features can also provide a latent space for regularizing an easily accessible atomic or sequence level representation to enhance transferability and enable an end-to-end machine learning model. Such a model can be particularly useful when limited experimental and/or quantum simulation data is available for a new molecular property.

While various processes for machine learning regression are described above, any variety of machine learning regression methods can be utilized in ML processes as appropriate to the requirements of specific applications in accordance with various embodiments of the invention including (but not limited to) ML processes that are trained using graph representations of quantum chemical information (see discussion above). MOB-ML processes that utilize clustering, regression and/or classification during training and/or evaluation in accordance with various embodiments of the invention are discussed further below.

### Clustering/Regression/Classification

Processes in accordance with various embodiments of the invention rely upon regression clustering, regression, and classification workflows for training and evaluating MOB-ML processes. As is discussed further below, any of a variety of workflows can be utilized as appropriate to the requirements of specific applications in accordance with various embodiments of the invention.

As the cost of GPR training scales cubically with the amount of data and becomes a computational bottleneck for large training sets, many embodiments implement clustering, regression, and/or classification steps into MOB-ML processes. In some embodiments, regression clustering (RC) can be used to partition the training data to best fit an ensemble of linear regression (LR) models. In several embodiments, each cluster can be regressed independently, using either LR or GPR. In yet some embodiments, a random forest classifier (RFC) can be trained for the determination of cluster assignments based on MOB feature values. RC recapitulates chemically intuitive groupings of the frontier molecular orbitals. Embodiments of MOB-ML processes including RC, LR, and RFC steps and RC, GPR, RFC steps can provide good prediction accuracy with greatly reduced wall-clock training times. In many embodiments, any of a variety of unsupervised and/or supervised clustering strategies can be utilized including (but not limited to) clustering on an embedded subspace and/or latent space. Furthermore, classification accuracy can be improved by applying different classifiers and soft clustering with different voting schemes. As can readily be appreciated, the specific clustering, regression and/or classification techniques that are utilized are largely only limited by the requirements of specific applications.

Many embodiments utilize RC to identify linear clusters and take advantage of the local linearity of pair correlation energies as a function of MOB features. Consider the set of M datapoints {fᵥ, εₜ}⊂ [symbol] ^{d}× [symbol] , where d can be the length of the MOB feature vector and where each datapoint can be indexed by t and corresponds to a MOB feature vector and the associated reference value (i.e., label) for the pair correlation energy. To separate these datapoints into locally linear clusters, S₁, . . . S_{N}, a solution can be used to the optimization problem in accordance with an embodiment:

$${min}_{{S}_{1} , … , {S}_{N}} {\sum }_{k = 1}^{N} {\sum }_{t ∈ {S}_{k}} {\vert A ( {S}_{K} ) \cdot {f}_{t} + b ( {S}_{k} ) - {ɛ}_{t} \vert}^{2} \qquad ( 9 )$$

where A(Sₖ) ∈ [symbol] ^{d} and b(Sₖ) ∈ [symbol] can be obtained via ordinary least squares (OLS) solution,

$$[ \begin{matrix}{f}_{t 1}^{T} & 1 \\ ⋮ & ⋮ \\ {f}_{{t}_{\vert {S}_{k} \vert}}^{T} & 1\end{matrix} ] [ \begin{matrix}A ( {S}_{k} ) \\ b ( {S}_{k} )\end{matrix} ] = [ \begin{matrix}{ɛ}_{t 1} \\ ⋮ \\ {ɛ}_{{t}_{\vert {S}_{k} \vert}}\end{matrix} ] \qquad ( 10 )$$

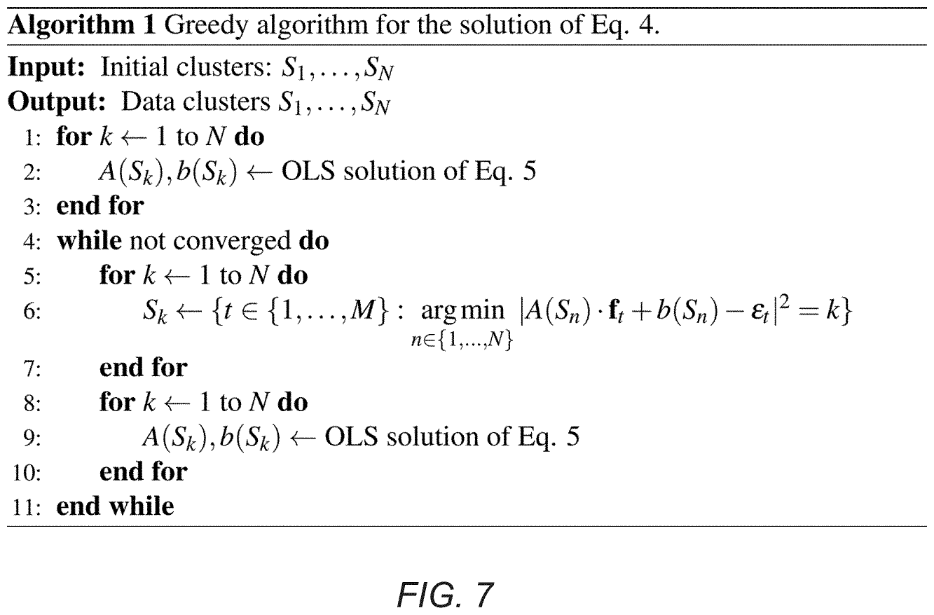

Each resulting Sₖ is the set of indices t assigned to cluster k comprised of |Sₖ| datapoints. A modified version of the greedy algorithm (FIG. 7) can be implemented to perform the optimization in Eq. 4 in some embodiments. Solutions to Eq. 4 may overlap, such that Sₖ ΩSₗ≠Ø for k≠l. The proposed algorithm can enforce clusters remain pairwise-disjoint.

Algorithm as shown in FIG. 7 has a per-iteration runtime of [symbol] (Md²), since we compute N OLS solutions each with runtime [symbol] (|Sₖ| d²) and since Σₖ₌₁^{N}|Sₖ|=m. However, the algorithm can be trivially parallelized to reach a runtime of [symbol] (max(|Sₖ|d²). A key operational step in this algorithm is line 6, which can be explained in simple terms as follows: each datapoint can be assigned, indexed by t, to the cluster to which it is closest, as measured by the squared linear regression distance metric,

|D_{n,t}|²=|A(Sₙ)·fₜ+b(Sₙ)−εEₜ|² (11)

where D_{n,t} is the distance of this point to cluster n. A datapoint can be equidistant to two or more different clusters by this metric; in such cases, the datapoint is randomly assigned to only one of those equidistant clusters to enforce the pairwise-disjointness of the resulting clusters. Convergence of the greedy algorithm can be measured by the decrease in the objective function of Eq. 9.

Processes in accordance with many embodiments rely upon regression clustering. RC can be performed using the ordinary least square linear regression implementation in the SCIKIT-LEARN package. The greedy algorithm can be initiated from the results of K-means clustering, also implemented in SCIKIT-LEARN in some embodiments. In several embodiments, K-means initialization can improve the subsequent training of the random forest classifier (RFC) in comparison to random initialization. In some embodiments, a convergence threshold of 1×10⁻⁸ kcal²/mol² for the loss function of the greedy algorithm (Eq. 9) can lead to no degradation in the final MOB-ML regression accuracy.

Processes in accordance with many embodiments rely upon regression. Some embodiments include ordinary least-squares linear regression (LR) as regression models. Several embodiments include Gaussian Process Regression (GPR) as regression models. In many embodiments, regression can be independently performed for the training data associated with each cluster, yielding a local regression model for each cluster. In several embodiments, regression can be independently performed for the diagonal and off-diagonal pair correlation energies (ε_{d}^{ML} and εₒ^{ML}) yielding independent regression models for each (Eq. 6). GPR can be performed using a negative log marginal likelihood objective.

Processes in accordance with many embodiments rely upon classification. RFC can be trained on MOB-ML features and cluster labels for a training set and then used to predict the cluster assignment of test datapoints in MOB-ML feature space in many embodiments. Some embodiments include the RFC implementation in SCIKIT-LEARN, using with 200 trees, the entropy split criteria, and balanced class weights. Several embodiments include alternative classifiers including (but not limited to) K-means, Linear SVM, and AdaBoost. As can readily be appreciated, the specific features used as classifiers are largely only limited to the requirements of specific applications.

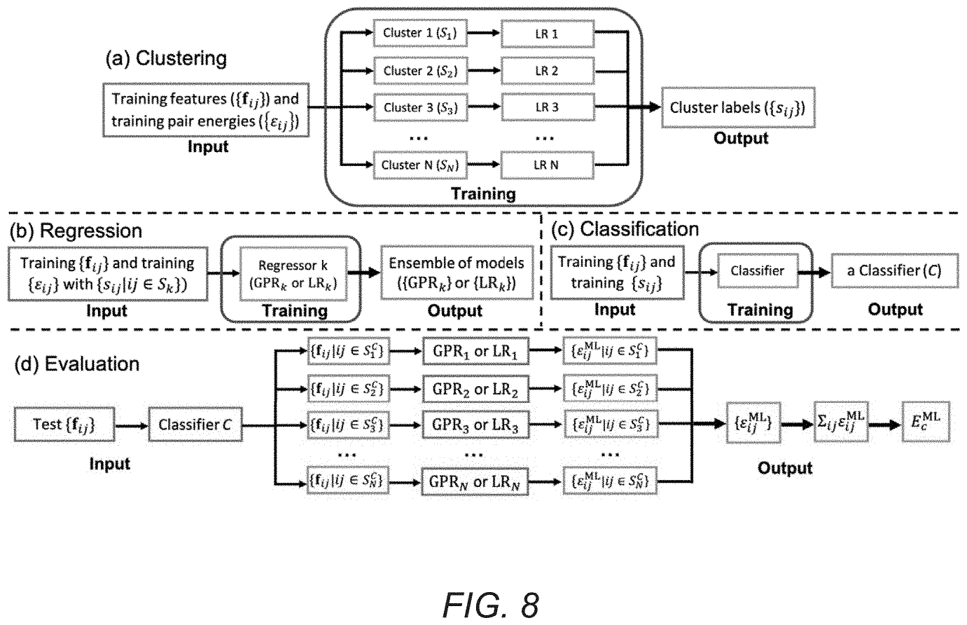

Processes in accordance with many embodiments of the invention rely upon a clustering/regression/classification workflow. FIG. 8 schematically illustrates workflow for training and evaluating of MOB-ML processes with clustering in accordance with an embodiment of the invention. The training can involve three steps. First, the training dataset of MOB-ML feature vectors and energy labels can be assigned to clusters using the RC method (FIG. 8A). Second, for each cluster of training data, the regression model (LR or GPR) can be trained to enable the prediction of pair correlation energies from MOB-ML vectors. Third, a classifier can be trained from the MOB-ML feature vectors and cluster labels for the training data, to enable the prediction of the cluster assignment from MOB-ML feature vectors.

In many embodiments, the resulting MOB-ML process can be specified in terms of the method of clustering (RC), the method of regression (either LR or GPR), and the method of classification (either RFC or the perfect classifier). A notation that specifies these options (e.g., RC/LR/RFC or RC/GPR/perfect) can be used to refer to a given MOB-ML process.

FIG. 8D schematically illustrates evaluation of the trained MOB-ML processes in accordance with an embodiment of the invention. A given molecule can be first decomposed into a set of test feature vectors associated with the pairs of occupied MOs. The classifier can be used to assign each feature vector to an associated cluster. The cluster-specific regression model can be used to predict the pair correlation energy from the MOB feature vector. Finally, the pair correlation energies can be summed to yield the total correlation energy for the molecule.

To improve the accuracy and reduce the uncertainty in MOB-ML processes, many embodiments include training of 10 independent ensembles of models using the clustering/regression/classification workflow. Several embodiments include computation by averaging over the 10 models and include the predictive mean and the corresponding standard error of the mean (SEM).

While various processes for regression clustering are described above, any variety of clustering methods can be utilized in MOB-ML processes as appropriate to the requirements of specific applications in accordance with various embodiments of the invention. Processes for molecular synthesis in accordance with various embodiments of the invention are discussed further below.

### Molecular Synthesis

Processes in accordance with various embodiments of the invention can be utilized to synthesize molecules. In several embodiments, MOB-ML processes are utilized to conduct a virtual screen of a set of candidate molecular systems based upon a set of one or more criteria related to chemical properties predicted by the MOB-ML model. In a number of embodiments, a molecular system is identified using an inverse design or generative process in which a search of a MOB feature space is performed based upon a set of one or more criteria related to a chemical properties predicted by the MOB-ML. Sets of MOB features that are predicted to possess desirable chemical properties by the MOB-ML model can then be utilized to identify molecular structures corresponding to the MOB features that are likely to possess the desired chemical properties. As is discussed further below, any of a variety of chemical property criteria can be utilized to perform virtual screening and/or inverse molecular design as appropriate to the requirements of specific applications in accordance with various embodiments of the invention.

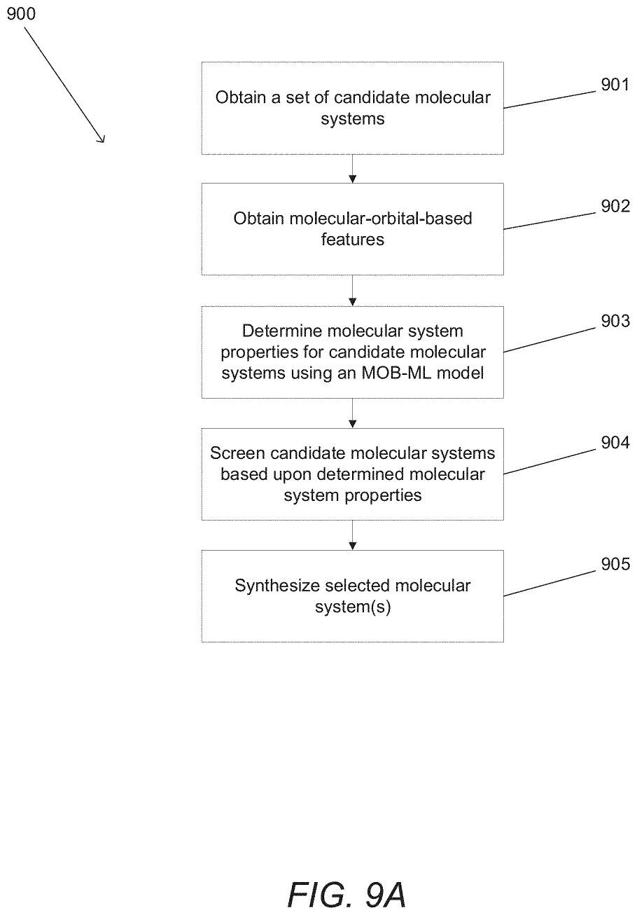

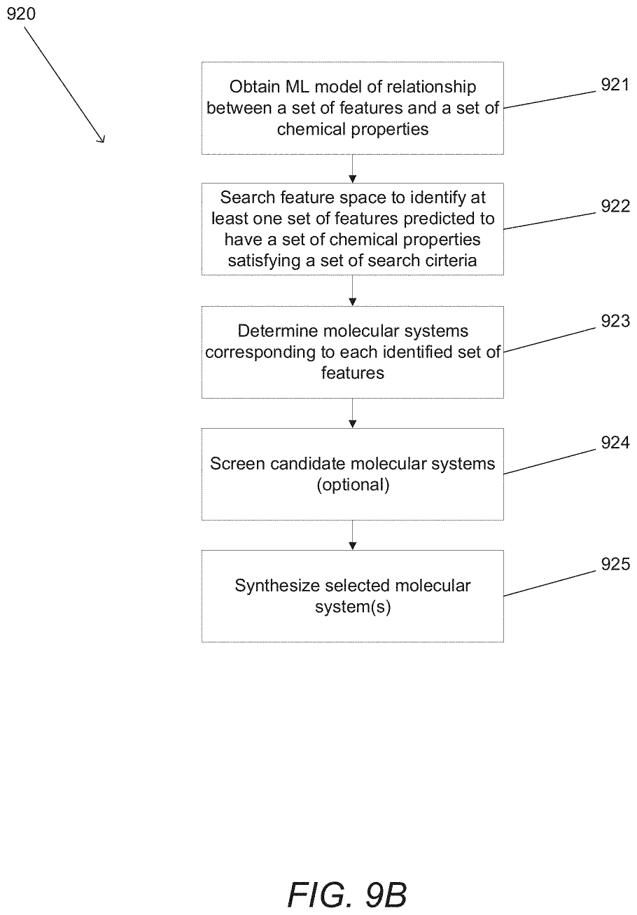

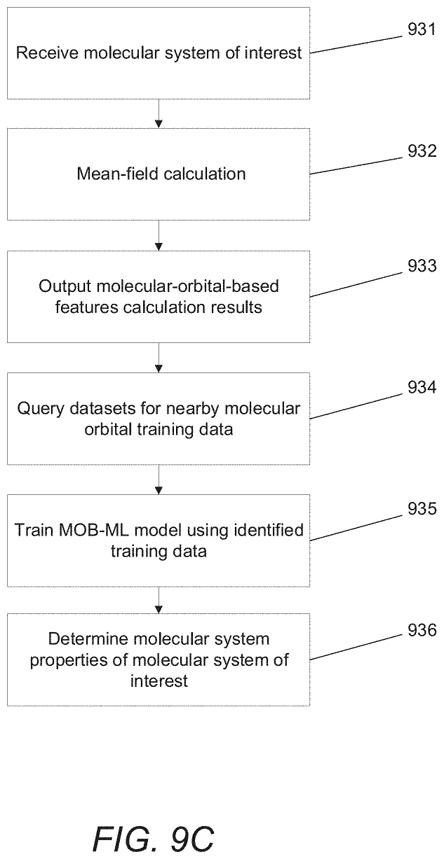

Many embodiments implement MOB-ML processes that screen a set of candidate molecular systems based upon a set of criteria related to one or more desirable chemical properties to identify a molecular structure to synthesize. A method for screening candidate molecular systems molecules using a MOB-ML process as part of a process for synthesizing a molecular system having a set of desirable characteristics in accordance with an embodiment of the invention is illustrated in FIG. 9A. The process 900 includes obtaining (901) a set of candidate molecular systems that are provided as inputs to the virtual screening process. In several embodiments, a quantum chemistry representation of the candidate molecular systems is obtained. In the illustrated embodiment, the candidate molecular systems are described (902) by a set of molecular-orbital-based features.

In several embodiments, an ML model that estimates one or more chemical properties based upon a quantum chemistry representation of a molecular system can be utilized in the virtual screening of the set of candidate molecular systems. In the illustrated embodiment, molecular system properties for the candidate molecular systems are predicted (903) using an MOB-ML model trained using a process similar to any of the various processes described above. As can readily be appreciated, the specific ML model depends largely upon the quantum chemistry representation utilized to represent the candidate molecular systems, any processes utilized to reduce the dimensionality of the feature space of the quantum chemistry representation, the specific chemical properties predicted by the ML model, and/or the requirements of specific applications.

Predicted chemical properties of candidate molecular systems can be utilized to screen the candidate molecular systems in accordance with one or more criteria related to a desirable set of molecular system chemical properties. In many embodiments, additional criteria can also be utilized as part of the screen including known chemical properties of particular molecular systems such as (but not limited to) water solubility and/or toxicity. In several embodiments, the synthesis process can also further optimize the chemical structure of an identified molecular system to further enhance one or more desirable chemical properties. As can readily be appreciated, decreasing an undesirable chemical property can be treated in an equivalent manner to increasing a desirable chemical property. The candidate molecular system(s) determined to satisfy the set of criteria of the screening process can be output as report information, and/or synthesized (905).

While many quantum chemistry ML processes utilize candidate molecular systems as a starting point, the process of training a ML model based upon feature vectors derived from quantum chemistry information can inherently define a feature space that can be used for inverse molecule design. Accordingly, systems and methods in accordance with many embodiments of the invention utilize a quantum chemistry feature space to identify sets of quantum chemistry features that are likely to result in a molecular system with desirable chemical properties, and then identify molecular systems corresponding to the identified set of quantum chemistry features.

A process for synthesizing a molecular system having a desired set of chemical properties identified using an inverse molecule design process in accordance with an embodiment of the invention is illustrated in FIG. 9B. The process 920 includes obtaining (921) a ML model that describes the relationship between a set of features and a set of chemical properties. As can readily be appreciated, an MOB-ML model can be utilized that is obtained using a process similar to any of the variety of processes for training MOB-ML models described above. In a number of embodiments, an ML trained based upon alternative quantum chemistry representations of molecular systems including (but not limited to) attributed graph representations can also be utilized. As can readily be appreciated, the specific ML model that is utilized depends largely upon the requirements of a particular application.

A search (922) can then be performed within the feature space of the ML model to identify sets of features that the ML model predicts will have a set of chemical properties that satisfy a set of search criteria. In a number of embodiments, the search can be conducted using a non-linear optimization process. In a number of embodiments, the search can be performed using a generative model such as (but not limited to) a variational autoencoder (VAE), a Generative Adversarial Network (GAN) and graph kernels. The generative models can be utilized to learn how to generate sets of features that successively improve upon the extent to which the ML model predicts that the generated sets of features satisfy the set of criterion of the search. As can readily be appreciated, any of a variety of techniques can be utilized to identify one or more sets of features within the feature space that a ML model predicts will have chemical properties satisfying a set of one or more chemical property criteria.

As can readily be appreciated, the feature space corresponds to quantum chemical representations of molecular systems. Therefore, the inverse molecular design process involves identification (923) of a molecular system possessing a quantum chemical representation corresponding to the identified set of features. In a number of embodiments, the mapping of a set of features in the feature space of the ML model to a molecular system can be achieved using a feature-structure map. In several embodiments, the feature-structure map can be learned from a set of training data in which molecular structures with bonding information and/or any other atomic representations are annotated with sets of features in the feature space. In a number of embodiments, the molecular structures can be represented as SMILES strings. As can readily be appreciated, any of a variety of training data sets and/or machine learning processes can be utilized to learn a process for mapping from a feature space to specific molecular structures.

In a number of embodiments, the inverse molecule design process yields a set of candidate molecular systems with predicted chemical properties. An addition screen can be performed (924) to filter the list of candidate molecular systems based upon a variety of criteria including (but not limited to): complexity of chemical synthesis, known toxicity, water solubility, and/or any of a variety of alternative chemical properties. When an appropriate candidate molecular system is identified, a report can be generated and/or the selected molecular system synthesized (925).

While various processes for identifying molecular structures for synthesis are described above, any of a variety of processes that identify molecular structures using ML models can be utilized to perform chemical synthesis as appropriate to the requirements of specific applications in accordance with various embodiments of the invention. ML processes can also be utilized in the context of quantum chemistry calculations for a variety of additional purposes. Processes for using ML in quantum chemistry calculations in accordance with various embodiments of the invention are discussed further below.

### Molecular “Fitting Room”

In a number of embodiments, a particular molecular system of interest can be utilized to identify a set of relevant molecular orbital training data from a database of molecular systems for which chemical properties are known. The database of molecular systems can be queried to identify molecular orbitals based upon distance in feature space between molecular orbitals represented within the database and molecular orbitals of the molecular system of interest. A distance metric can be utilized to measure the distance between MOB features of the molecular orbitals in the database and the MOB features of the molecular orbitals of the molecular system of interest. In this way, a molecular system specific training data set can be generated for the purposes of training an MOB-ML model to predict the chemical properties (e.g. quantum mechanical energy) of the molecular system of interest.

A specific process for generating a MOB-ML for estimating the chemical properties of a specific candidate molecular system in accordance with an embodiment of the invention is illustrated in FIG. 9C. The MOB-ML process receives (931) as an input a specific molecular system. A set of MOB features for the molecular orbitals of the specific molecular system are generated. In the illustrated embodiment, the MOB features are generated by performing (932) mean-field calculations and obtaining (933) MOB features based upon the results of the calculations. The MOB features can then be utilized to query (934) a database to identify molecular orbitals that are described within the database that are proximate in MOB feature space to the molecular orbitals of the specific molecular system of interest. The MOB features of the proximate molecular orbitals and their chemical properties can then be utilized to train (935) an MOB-ML model that can then be utilized to accurately predict (936) the chemical properties of the specific molecular system that was the input of the process. As can readily be appreciated, training MOB-ML models in the specific region in feature space occupied by a particular specific molecular system can greatly increase the accuracy with which estimates can be made of the chemical properties of that specific molecular system.

While the discussion of the processes described above with reference to FIG. 9C largely focus on processes for identifying training data in a MOB feature space, similar processes can be performed using any of a variety of MOB representations of molecular systems including (but not limited to) attributed graph representations. Systems and methods that utilize ML processes and ML models similar to those described above to provide quantum chemistry calculations for specific molecular systems are discussed further below.

### Quantum Chemistry Program

Processes in accordance with various embodiments of the invention rely upon quantum chemistry properties. As is discussed further below, any of a variety of quantum chemistry predictions of MOB features of different molecular systems can be utilized as appropriate to the requirements of specific applications in accordance with various embodiments of the invention.

Many embodiments implement physics-based quantum chemistry predictions as input MOB features of molecular systems during MOB-ML processes. Several embodiments implement predictions of physics-based quantum chemistry for the molecular system on the basis of MOB features. Some embodiments include that the output results can include molecular system properties. Various embodiments of physics-based quantum chemistry programs include (but are not limited to) coupled-cluster theory and MP2 theory. As can readily be appreciated, the specific features used as quantum chemistry programs are largely only limited to the requirements of specific applications. Many embodiments are incorporated in software packages.

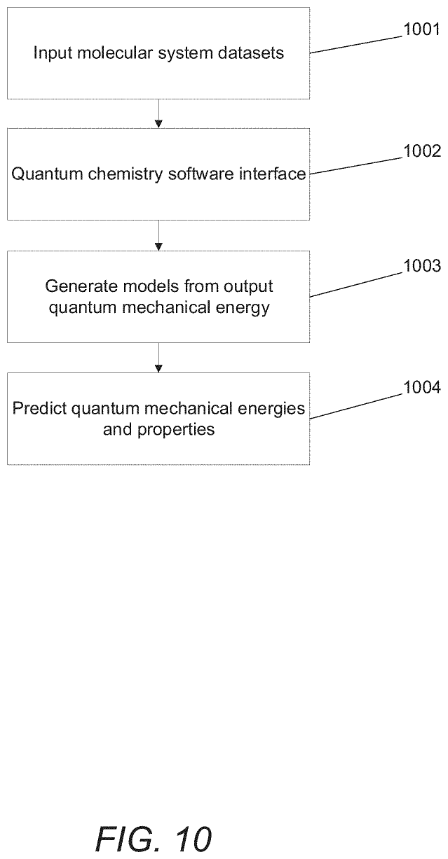

A system for incorporating an MOB-ML process into a software package in accordance with an embodiment of the invention is illustrated in FIG. 10. A user can provide input to a quantum chemistry software package 1001. The user can perform physics-based calculations 1002. Results of the calculations can be replaced with the predictions of a ML model from the MOB features corresponding to the user inputs 1003. Generalizations can include accelerating rather than replacing physics-based calculations using models based on MOB features to predict intermediate quantities 1004; and generation of the machine learned model using these strategies.

In some embodiments, software packages incorporating MOB-ML processes can be operated on a user-friendly platform, examples of such embodiments include (but are not limited to): smart phones, tablets, and computers. As can readily be appreciated, the specific features used as user platforms are largely only limited to the requirements of specific applications. According to some embodiments, the software package performs quantum simulations in seconds via a backend cloud-based deployment of MOB-ML processes.

While various processes for generating quantum chemistry predictions from MOB features are described above, any variety of processes that predict molecular system properties based on MOB features can be utilized in MOB-ML processes as appropriate to the requirements of specific applications in accordance with various embodiments of the invention. Various examples implementing MOB-ML processes in accordance with various embodiments of the invention are discussed further below.

### EXEMPLARY EMBODIMENTS

The following section provides specific examples of the use of different MOB-ML processes to determine molecular compositions and structures for synthesis. The features and training pair energies associated with the various geometries discussed below can be computed using the MOLPRO 2018.0 software package in a cc-pVTZ basis set for small molecule systems (see Examples 1-6 below) and for big molecules (e.g. QM7b-T and GDB-13-T) an MP2/cc-pVTZ and/or CCSD(T)/cc-pVDZ can be utilized. Localized molecular orbitals used in feature construction can be determined using an Intrinsic Bond Orbital method for both occupied and virtual space (see Examples 2, 5 and 6 below), or using the Boys method for occupied space and the Intrinsic Bond Orbital method for virtual space (see remaining examples below). Reference pair correlation energies can be computed with second-order MP2 theory and CCSD theory as well as with perturbative triples CCSD(T). Density fitting for both Coulomb and exchange integrals are employed for some of the results presented below (see Example 7 below which uses density fitting for QM7b-T and GDB13-T). The frozen core approximation can also be utilized.

Gaussian process regression (GPR) can be employed to machine learn ε_{d}^{ML} and εₒ^{ML} (Eq. 6) using the GPY 1.9.6 software package. The GPR kernel is Matérn 5/2 with white noise regularization. Kernel hyperparameters can be optimized with respect to the log marginal likelihood objective for the alkane series results, as well as for ε_{d}^{ML} of the QM7b results. The Matérn 3/2 kernel instead of the Matérn 5/2 kernel can be used for the case of εₒ^{ML} for QM7b-T results.

Feature selection can be performed using the random forest regression implementation in the SCIKIT-LEARN v0.20.0 package with a mean decrease of accuracy importance criteria.

Training and test geometries can be sampled at 50 fs intervals from ab initio molecular dynamics trajectories performed with the Q-CHEM 5.0 software package, using the B3LYP/6-31g* level of theory and a Langevin thermostat at 350 K.

As can readily be appreciated, MOB-ML processes can be implemented in any of a variety of different ways and/or using any of a variety of different software packages. It will be understood that the specific embodiments are provided for exemplary purposes and are not limiting to the overall scope of the disclosure, which must be considered in light of the entire specification, figures and claims.

### Example 1: Determination of CCSD and MP2 Correlation Energies of Water Molecule

Many embodiments implement transferability of MOB-ML process among molecular geometries. Several embodiments include the determination of correlation energies of water molecule geometries based on MOB-ML processes trained on pair energies from randomly sampled water molecule geometries.

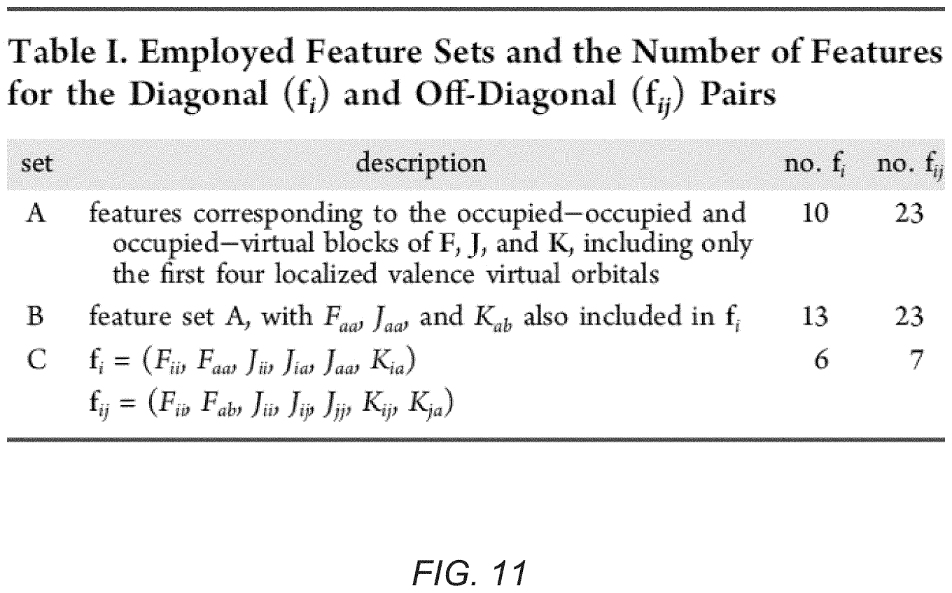

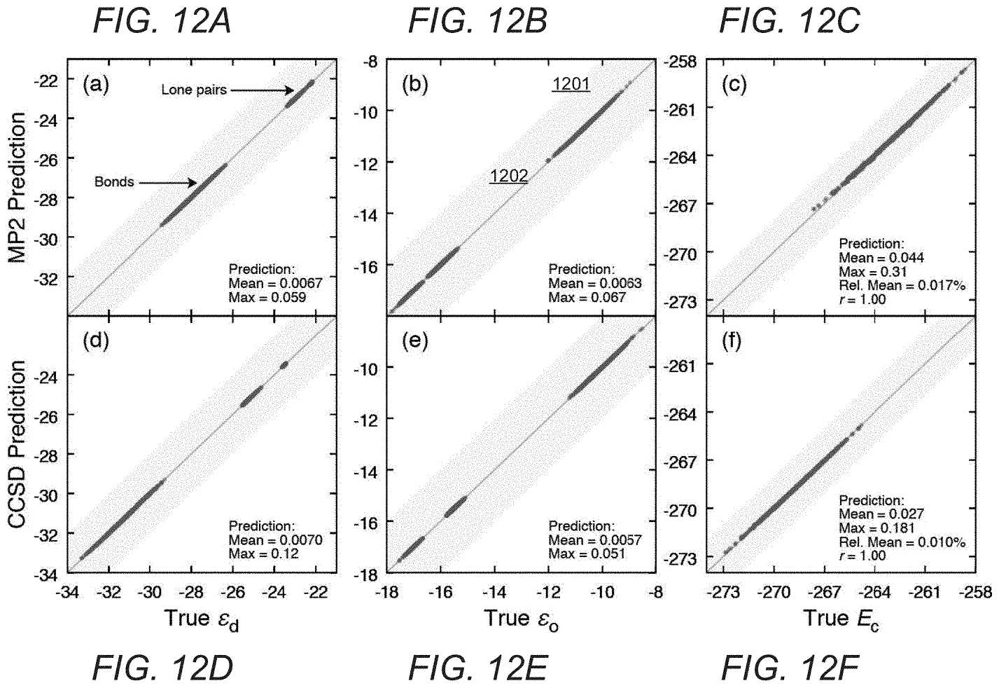

In some embodiments, MOB-ML processes include a single water molecule on a subset of geometries to predict the correlation energy at other geometries. For both the Møller-Plessett perturbation theory (MP2) and coupled-cluster with singles and doubles (CCSD) levels of theory, the diagonal (ε_{d} and ε_{d}^{ML} are used interchangeably) and off-diagonal (εₒ and εₒ^{ML} are used interchangeably) contributions to the correlation energy can be separately trained using feature set A, as listed in FIG. 11, with 200 training geometries, and the resulting predictions for a superset of 1000 geometries are presented in FIG. 12. FIG. 11 includes employed feature sets and the number of features for the diagonal (fᵢ) and off-diagonal (fᵢⱼ) pairs. Errors are summarized in terms of mean absolute error (mean error), maximum absolute error (max error), and mean error as a percentage of the mean total correlation energy (rel mean error). Energies are reported in milliHartrees (mH). The Pearson correlation coefficient (r) is also reported as a measure of correlation between the MOB-ML process predictions and the true values; a value of r=1 indicates perfect correlation, r=0 indicates no correlation, and r=−1 indicates a perfect anticorrelation. Note that a value of r=1 does not imply that the slope of the relationship is unity.

The MOB-ML prediction results for a single water molecule training on 200 geometries and predictions for 1000 geometries are shown in FIG. 12A-12C of MP2 correlation energy and in FIG. 12D-12F of CCSD correlation energy, including ε_{d} (FIG. 12A, 12D) and εₒ (FIG. 12B, 12E) for the pairs of occupied orbitals, as well as the total correlation energies (FIG. 12E, 12F). Mean absolute errors (mean), maximum absolute errors (max), mean errors as a fraction of total correlation energy (rel mean), and the Pearson correlation coefficient (r) are illustrated. The guideline 1202 indicates zero error, with the region of up to 2 mH error indicated via shading 1201. As illustrated for the diagonal contributions in FIG. 12A, the individual contributions to the correlation energy exhibit clusters associated with common physical origins (i.e., σ-bonding vs lone-pair orbitals). For both the diagonal and off-diagonal contributions, the agreement between the MOB-ML prediction and the reference result is high, which can lead to predictions for the total correlation energy that are well within chemical accuracy. The quality of MOB-ML processes for MP2 and CCSD are qualitatively similar (FIG. 12).

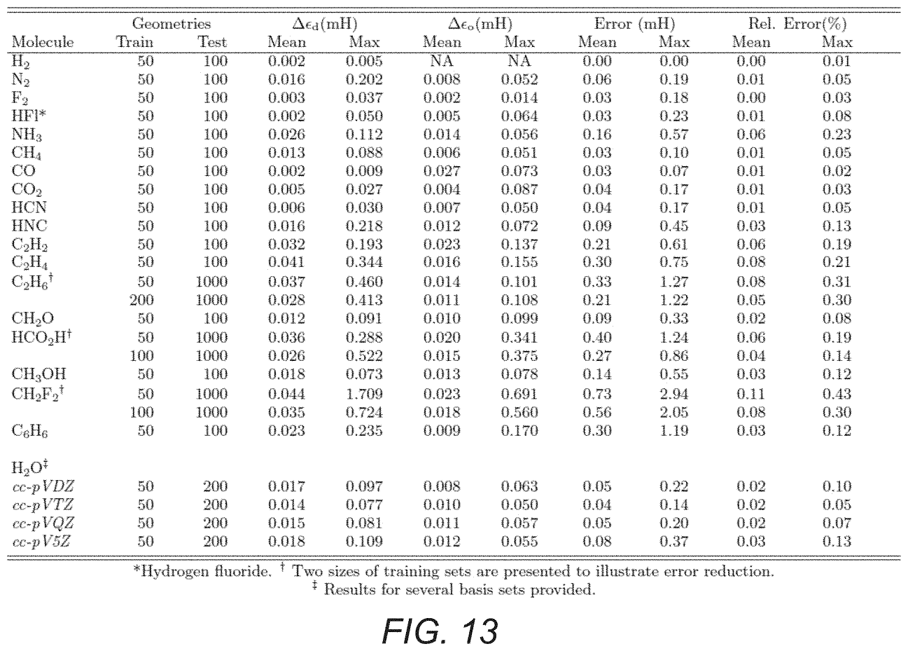

FIG. 13 summarizes the corresponding results for other small molecules, with ε_{d} and εₒ trained on a subset of geometries and used to predict the CCSD correlation energy for other geometries. FIG. 13 includes detailed decomposition of MOB-ML predictions of CCSD correlation energies for the collection of small molecules, with the number of training and testing geometries indicated. Mean and Max Errors are reported for the diagonal (ΔE_{d}) and off-diagonal (ΔEₒ) contributions to the correlation energy, as well as the corresponding total and relative correlation energy errors.

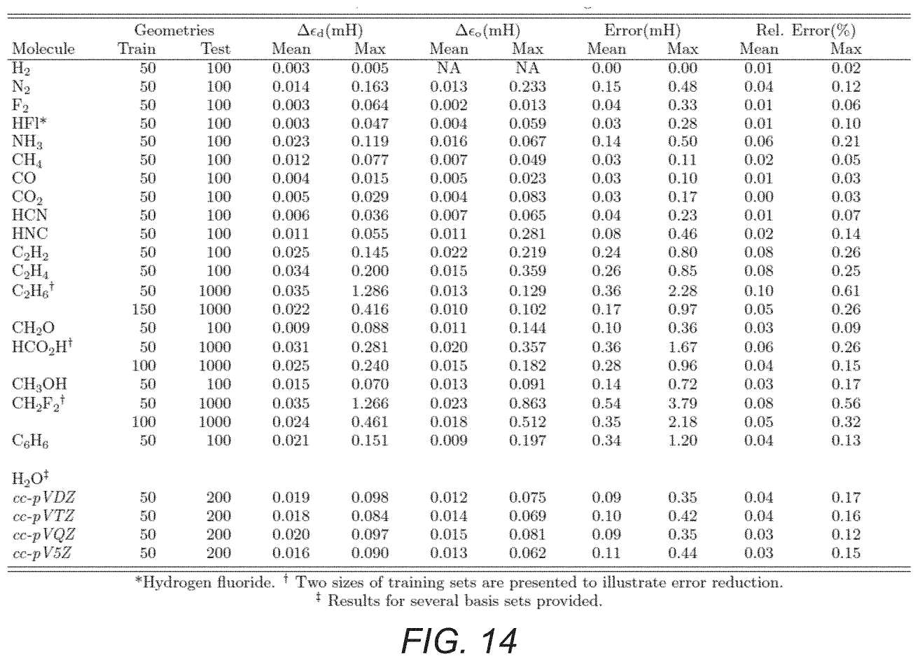

FIG. 14 summarizes the corresponding results for other small molecules, with ε_{d} and εₒ trained on a subset of geometries and used to predict the MP2 correlation energy for other geometries. The molecules range in size from H₂ to benzene. Feature set A is used in all cases, except for ethane, for which feature set B is utilized to achieve comparable accuracy. The number of geometries included in the training set and testing superset are indicated in the tables. In general, the mean error for the correlation energy is much less than 1 mH, and the max error is also in the range of chemical accuracy. The MOB-ML processes predicting the correlation energy for these molecules with a rel mean error that is at most 0.1% for all listed molecules. FIG. 14 includes detailed decomposition of MOB-ML predictions of MP2 correlation energies for the collection of small molecules, with the number of training and testing geometries indicated. Mean and max errors are reported for the diagonal (Δε_{d}) and off-diagonal (Δε₀) contributions to the correlation energy, as well as the corresponding total and relative correlation energy errors.

FIG. 13 and FIG. 14 also show the sensitivity of the MOB-ML predictions to changing the number of geometries in the training set (for ethane, formic acid, and difluoromethane) or the employed basis set (for water). The water results for basis sets ranging from double-ζ to quintuple-ζ make clear that the ML prediction is not sensitive to the employed basis set.

In some embodiments, a separate MOB-ML process can be trained to predict the correlation energy at the MP2, CCSD, and CCSD(T) levels of theory, using reference calculations on a subset of 1000 randomized water geometries to predict the correlation energy for the remainder. Feature selection with an importance threshold of 1×10⁻³ results in 12, 11 and 10 features for εₒ^{ML} for MP2, CCSD and CCSD(T), respectively; ten features are selected for ε_{d}^{ML} for all three post-Hartree-Fock methods.

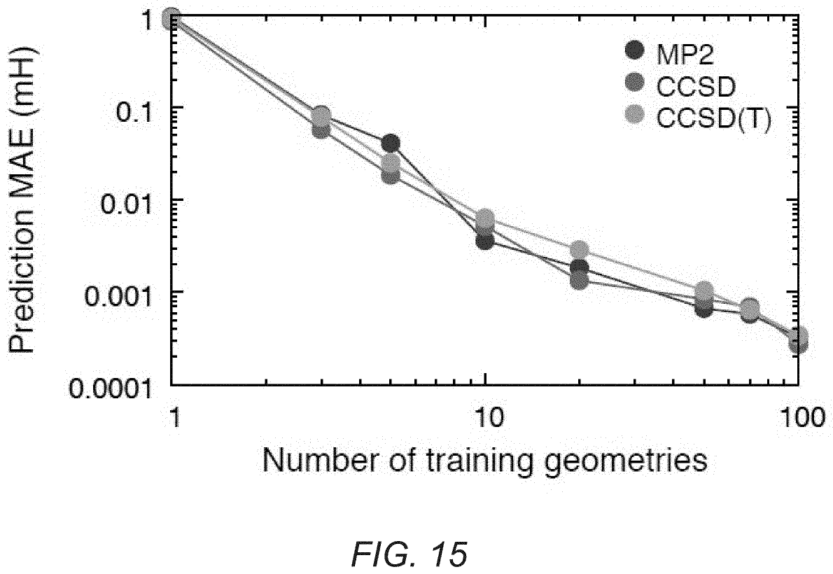

FIG. 15 presents the test set prediction accuracy of the MOB-ML processes as a function of the number of training geometries (for example, the learning curve) in accordance with an embodiment. MOB-ML predictions are shown for MP2, CCSD, and CCSD(T), and the MOB-ML process shows the same level of accuracy for all three methods. All three models achieve a prediction mean absolute error (MAE) of 1 mH when trained on a single water geometry, indicating that only a single reference calculation is needed to provide chemical accuracy for the remaining 999 geometries at each level of theory. Since it contains 10 distinct LMO pairs, this single geometry can provide enough information to yield a chemically accurate MOB-ML process for the global thermally accessible potential energy surface.

For all three methods shown in FIG. 15, the learning curve exhibits the power-law behavior as a function of training data, and the total error reaches microhartree accuracy with tens of water training geometries. In many embodiments, the prediction accuracy of the MOB-ML processes is based on the use of Boys localization, which can specify unique and consistent LMOs corresponding to the oxygen lone pairs.

### Example 2: Determination of CCSD and MP2 Correlation Energies of Water Clusters

Many embodiments implement MOB-ML process transferability within a molecular family. For example, several embodiments include determination of CCSD and MP2 correlation energies of water clusters based on MOB-ML training on water monomers and dimers.

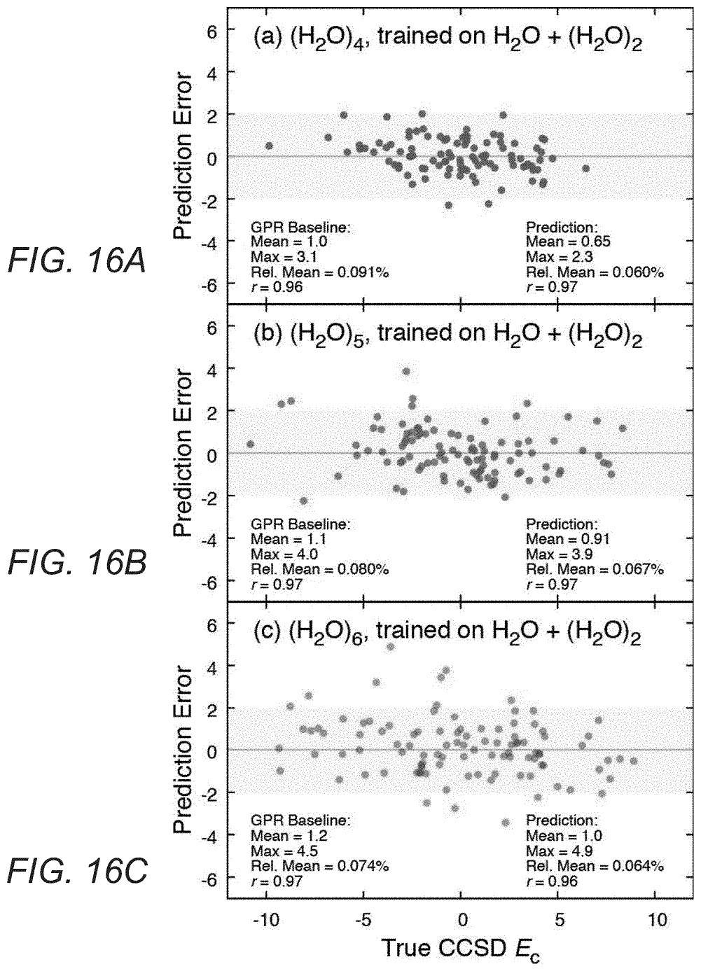

In one embodiment, FIG. 16 shows MOB-ML process prediction results of CCSD correlation energies for water clusters: FIG. 16A of tetramer, FIG. 16B of pentamer, FIG. 16C of hexamer, based on training data that include water monomer and dimer. The MOB-ML process can be trained on 200 water monomer and 300 water dimer geometries, and correlation energy predictions can be made for 100 geometries of each of the larger water clusters. MOB-ML prediction errors are plotted versus the true CCSD correlation energy. Parallelity error is removed via a global shift in the predicted energies of the tetramer, pentamer, and hexamer by 1.7, 2.1, and 3.2 mH, respectively. GPR baseline errors correspond to the self-training error of the MOB-ML processes, providing an expectation for the lowest possible error of the model obtained from training on water monomer and dimer geometries. The true CCSD energies are plotted relative to their median. Energies are reported in mH. For the three clusters, the observed rel mean errors of 0.06-0.07% are comparable to those reported in Table II, and the Pearson correlation coefficients exceed 0.95.

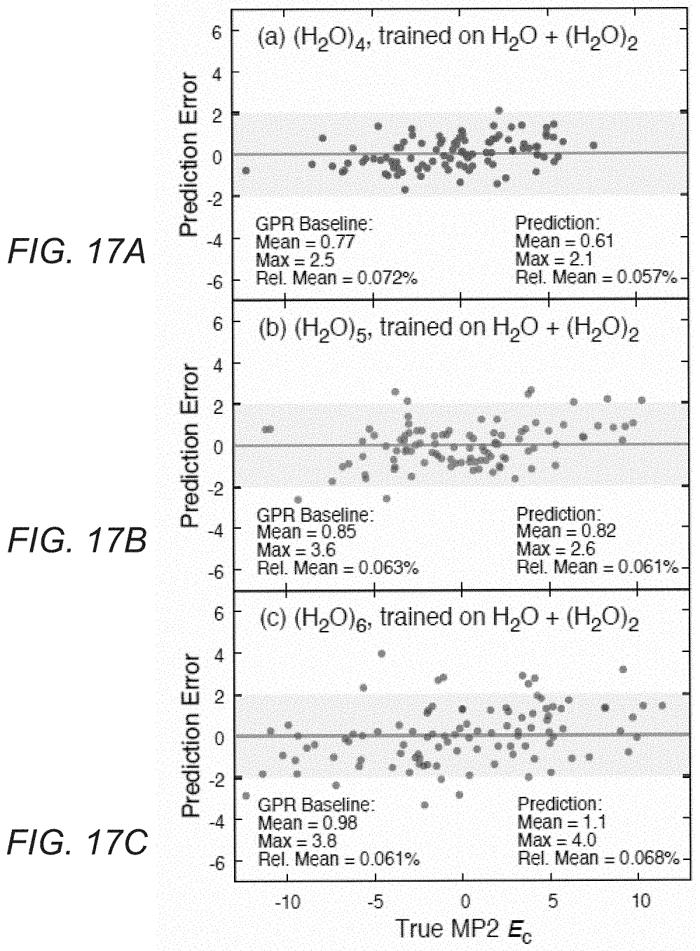

In another embodiment, FIG. 17 shows MOB-ML process prediction results of MP2 correlation energies for water clusters: FIG. 17A of tetramer, FIG. 17B of pentamer, FIG. 17C of hexamer, based on training data that include water monomer and dimer. The MOB-ML process can be trained on 200 water monomer and 300 water dimer geometries, and molecular structure predictions can be made for 100 geometries of each of the larger clusters. MOB-ML prediction errors are plotted versus the true MP2 correlation energy. Parallelity error is removed via a global shift in the predicted energies of the tetramer, pentamer, and hexamer by 0.68, 0.40, and 0.38 mH, respectively. Energies are reported in mH. For the three clusters, the observed rel mean errors of 0.06-0.07% are comparable to those reported in Table II.

FIGS. 16 and 17 show the calculated MOB-ML process baseline accuracy, determined via characterizing the self-training error with the employed MOB-ML process. For each size of water cluster, MOB-ML processes can be trained and tested on the same set of 100 geometries. Such embodiments establish the smallest error that can be expected of the predictions within the framework which can maximize model likelihood rather than minimizing training error. The prediction errors for the MOB-ML processes for the water clusters are very similar to the MOB-ML process baseline errors in both FIGS. 16 and 17, as the prediction error is dominated by the self-training error of the MOB-ML process rather than from a lack of transferability of the MOB-ML processes trained on water monomers and dimers to larger clusters.

### Example 3: Determination of CCSD and MP2 Correlation Energies of Butane and Isobutane

Many embodiments implement MOB-ML process transferability within a molecular family of covalently bonded molecules. Several embodiments include determination of CCSD and MP2 correlation energies of butane and isobutane based on MOB-ML training of shorter alkane datasets.

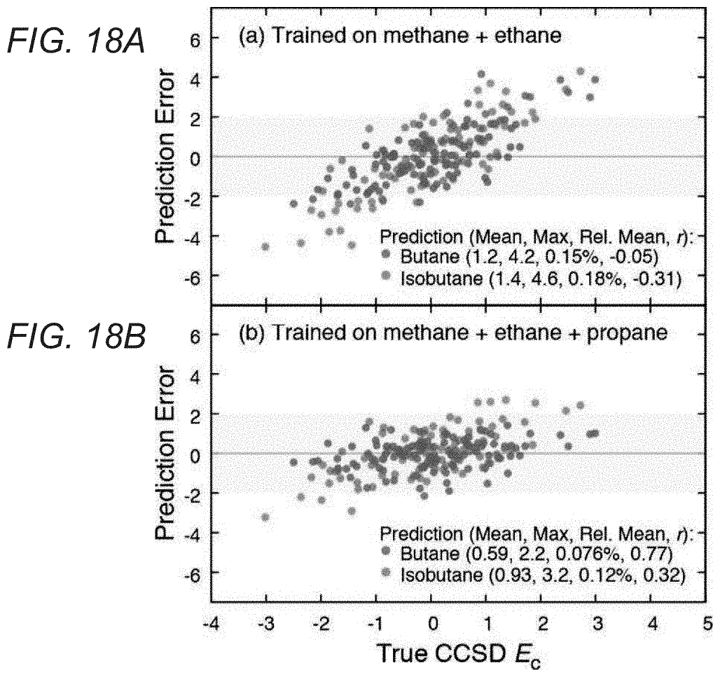

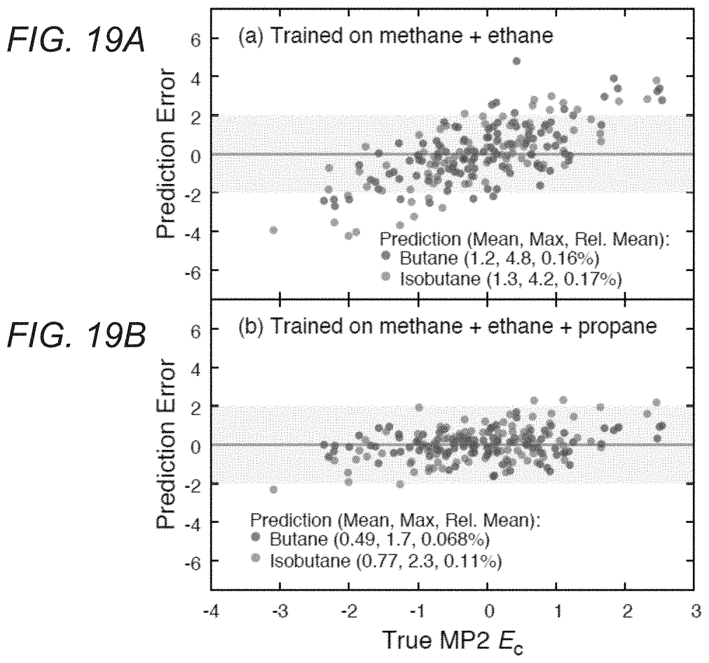

MOB-ML processes in accordance with many embodiments of the invention can be trained on 100 methane and 300 ethane geometries using feature set B as shown in FIG. 11. In some embodiments, FIGS. 18A, 18B and 19 present the resulting MOB-ML predictions for 100 geometries of butane and isobutane. FIGS. 18A and 18B show CCSD correlation energies for butane and isobutane, with MOB-ML processes obtained from training on methane and ethane in FIG. 18A, and methane, ethane and propane in FIG. 18B. Prediction errors are plotted versus the true CCSD correlation energy. Parallelity error is removed via a global shift in the predicted energies of butane and isobutane by 25 and 16 mH (FIG. 18A), and 3.3 and 0.73 mH (FIG. 18B) respectively. The mean and max MOB-ML baseline errors for butane are 0.58 and 1.5 mH, respectively. For isobutane, the errors are 0.53 and 1.9 mH. The MOB-ML baseline Pearson correlation coefficients for butane and isobutane are both 0.79. The true CCSD energies are plotted relative to their median. All energies are reported in mH.

FIG. 19 shows MP2 correlation energies for butane and isobutane, with MOB-ML processes obtained from training on methane and ethane in FIG. 19A, and methane, ethane and propane in FIG. 19B. Prediction errors are plotted versus the true CCSD correlation energy. Parallelity error is removed via a global shift in the predicted energies of butane and isobutane by 32 and 21 mH (FIG. 19A), and 3.3 and 0.87 mH (FIG. 19B) respectively. The mean and max MOB-ML baseline errors for butane are 0.40 and 1.2 mH, respectively. For isobutane, the errors are 0.47 and 1.7 mH. The true CCSD energies are plotted relative to their median. All energies are reported in mH.

The mean errors of CCSD correlation energies prediction are not large (1.2 and 1.4 mH) as shown in FIG. 18A. The rel mean errors are over twice those obtained for the water cluster series, and the mean and max errors associated with the baseline MOB-ML accuracy are smaller than the prediction errors. Moreover, the correlation coefficients are significantly reduced (−0.05 and −0.31) compared the water clusters described above in FIG. 16 and FIG. 17.

The effect of including additional alkane training data in FIG. 18B presents the MOB-ML model is retrained with the training data set expanded to include 50 propane geometries. The prediction errors and correlation coefficients for butane and isobutane can be substantially improved upon inclusion of the propane data, with the butane prediction errors dropping to the MOB-ML baseline while the isobutane prediction errors remain above the MOB-ML baseline. Specifically, the correlation coefficients increase to 0.77 and 0.32 for butane and isobutane, respectively, as compared to a MOB-ML baseline correlation coefficient of 0.79 for both molecules.

Many embodiments include different carbon atom-types included in the training data reflect the differences of the MOB-ML prediction errors in FIGS. 18A and 18B. The unbranched butane molecule includes only primary and secondary carbons, whereas isobutane includes a tertiary carbon atom. In FIG. 18A, the training datasets do not include examples of secondary or tertiary carbon atoms. The prediction results for butane and isobutane, both of which include atom-types that are not included in the training datasets, have small mean errors of 1.2 and 1.4 mH. In FIG. 14B, the propane training datasets can provide information about secondary carbons to the particular benefit of the butane predictions, which results butane prediction mean error of 0.59 mH. Whereas the isobutane errors, while improved at 0.93 mH, remain slightly larger since tertiary carbon examples are still not included in the training datasets. Many embodiments include that the MOB-ML processes exhibit transferability and provide good prediction accuracy even for molecules with atom-types that are not included in the training datasets.

### Example 4: Determination of CCSD(T) Correlation Energies of n-Butane and Isobutane

Many embodiments implement MOB-ML process transferability within a molecular family of covalently bonded molecules. Several embodiments include determination of CCSD(T) correlation energies of larger and more branched n-butane and isobutane based on MOB-ML model trained on thermalized geometries of shorter alkane datasets.

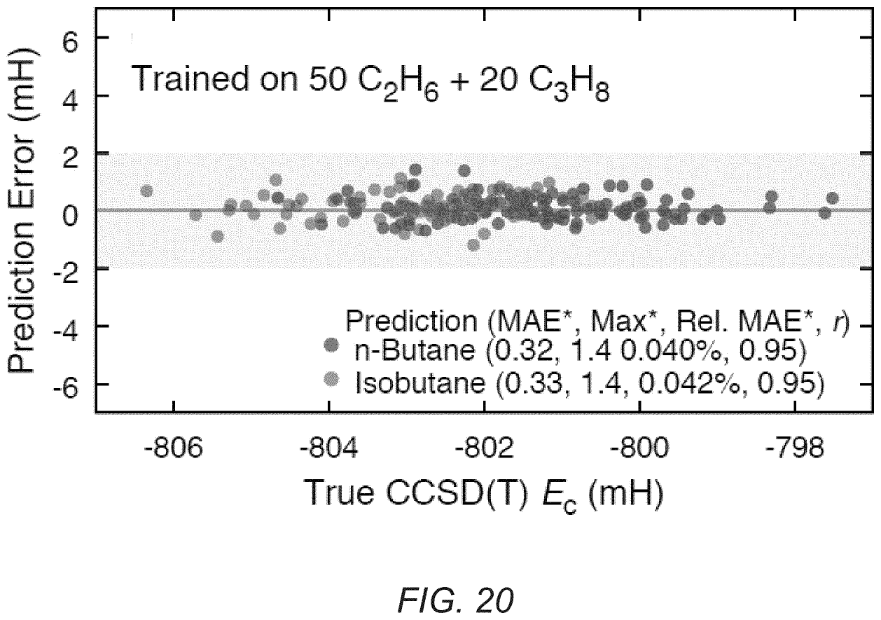

FIG. 20 shows MOB-ML predictions of the correlation energy for 100 n-butane and isobutane geometries in accordance to an embodiment. Training sets include 50 ethane and 20 propane geometries. MOB-ML prediction errors are plotted versus the true CCSD(T) correlation energy. To remove parallelity error, a global shift is applied to the predictions of n-butane and isobutane by 0.90 and 0.17 mH, respectively. Summary statistics including this shift (indicated by an asterisk) include: mean absolute error (MAE*), maximum absolute error (Max*), MAE* as a percentage of Ec (Rel. MAE*), and Pearson correlation coefficient (r). The gray shaded region corresponds to errors of ±2 mH.

In the predictions of MOB-ML shown in FIG. 20, the mean errors of n-butane and isobutane can be determined to be 0.32 mH and 0.33 mH and have with nearly identical accuracy. The prediction errors are not skewed as a function of true correlation energy. The primary methodological sources of these improvements are found to be symmetrization of occupied orbitals (Eq. 7) and the improved feature selection methodology. The MOB-ML features can be selected with an importance threshold of 1×10⁻⁴, resulting in 27 features for ε_{d}^{ML} and 12 features for εₒ^{ML}.

### Example 5: Determination of CCSD and MP2 Correlation Energies of Methanol Using an MOB-ML Model Trained with Respect to Water, Methane, and Formic Acid

Many embodiments implement MOB-ML process transferability across molecules and elements. Several embodiments include determination of CCSD and MP2 correlation energies of water, methane, formic acid, and methanol based on MOB-ML training of water, methane, and formic acid.

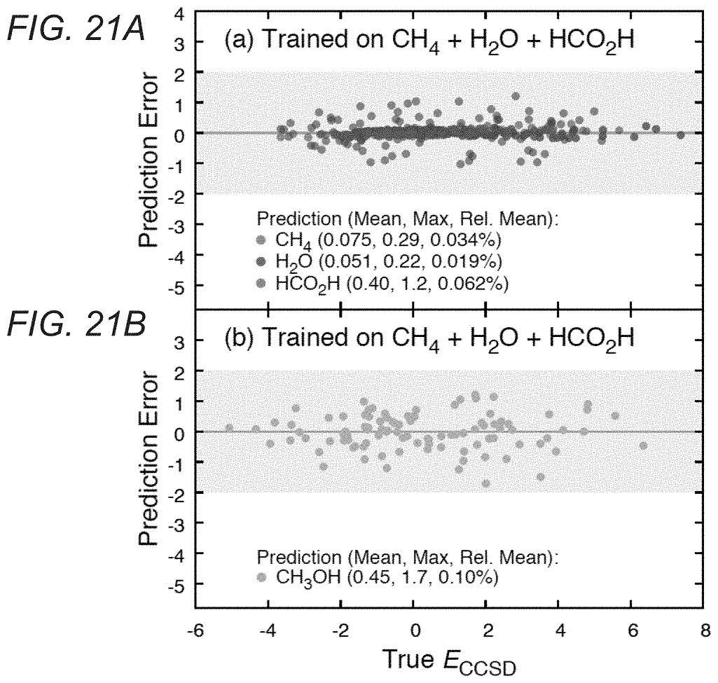

FIGS. 21A and 21B show MOB-ML predictions for methanol using a training set that contains methane, water, and formic acid in accordance with an embodiment. The training molecules can include similar bond-types and the same elements as methanol, but different bonding connectivity. The MOB-ML model is trained on 50 geometries each of methane, water, and formic acid, using Feature Set A in FIG. 11. The model can be used to predict CCSD energies for a superset of 100 geometries of each of the molecules in the training set in FIG. 21A and for 100 geometries of the methanol molecule in FIG. 21B. Prediction errors are plotted versus the true CCSD total energy. In FIG. 21B, parallelity error is removed via a global shift in the predicted energy by 3.5 mH. The true CCSD energies are plotted relative to their median. Energies reported in mH.

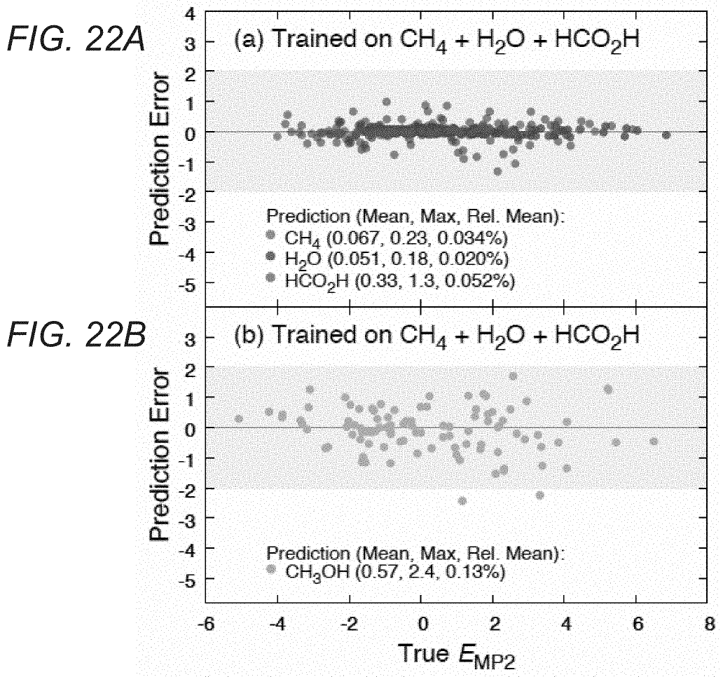

FIGS. 22A and 22B show MOB-ML predictions of MP2 total energy using a training set that contains methane, water, and formic acid for water, methane, and formic acid in FIG. 22A, and for methanol in FIG. 22B in accordance to an embodiment. The training molecules can include similar bond-types and the same elements as methanol, but different bonding connectivity. ML prediction errors are plotted versus the true CCSD total energy. In FIG. 22B, parallelity error is removed via a global shift in the predicted energy by 4.5 mH. The true CCSD energies are plotted relative to their median. Energies reported in mH.

FIGS. 21A and 22A first show predictions for the molecules that are represented within the training set. The resulting errors are similar to those observed when separate models are trained for each of these molecules individually (FIG. 13), indicating that the MOB-ML model can have the flexibility to simultaneously describe this group of chemically distinct molecules.

In FIGS. 21B and 22B, the same MOB-ML model can be used to predict the CCSD and MP2 energies of methanol, which is not represented in the training set. The resulting Mean and Max Errors for methanol can be comparable to those for the molecules in the training set. These errors are only about twice as large as those obtained from training methanol on itself (FIG. 13). Many embodiments include that the MOB-ML processes can be used to transfer information learned about pair correlation energies in methane, water, and formic acid toward the prediction of methanol, while preserving chemical accuracy.

### Example 6: Determination of CCSD and MP2 Correlation Energies of Ammonia, Methane, and Hydrogen Fluoride

Many embodiments implement MOB-ML process transferability across molecules and elements. Several embodiments include determination of CCSD and MP2 correlation energies of ammonia, methane, and hydrogen fluoride based on MOB-ML training of water.

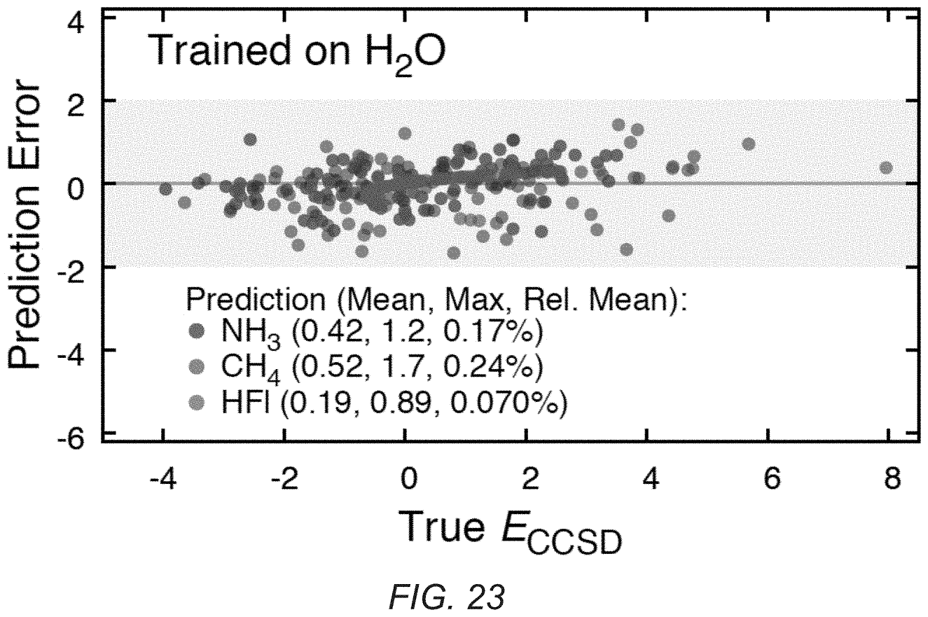

In an embodiment, FIG. 23 shows MOB-ML predictions for the CCSD energies of 100 geometries each of ammonia (NH₃), methane (CH₄), and hydrogen fluoride (HF), using MOB-ML processes trained on 100 water geometries. Feature Set C can be used to avoid over fitting. MOB-ML prediction errors are plotted versus the true CCSD total energy. Parallelity error is removed via a global shift in the predicted energies of ammonia, methane, and hydrogen fluoride by 3.4, 16, and 5.6 mH, respectively. The true CCSD energies are plotted relative to their median. Energies are reported in mH.

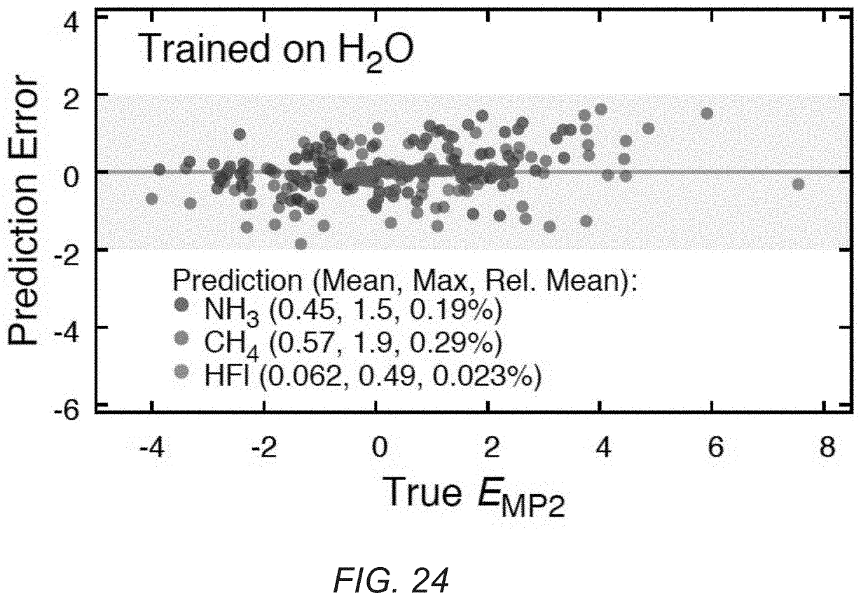

In another embodiment, FIG. 24 shows MOB-ML predictions for the MP2 energies of 100 geometries each of ammonia, methane, and HF, using MOB-ML processes trained on 100 water geometries. Parallelity error is removed via a global shift in the predicted energies of ammonia, methane, and hydrogen fluoride by 24, 51, and 12 mH, respectively.

FIG. 23 shows that the CCSD energies for the NH₃, CH₄, and HF molecules can be accurately predicted by the MOB-ML processes on the basis of training data that comes entirely from H₂O. The Mean Errors fall within 0.5 mH, and Rel. Mean Errors remain below 0.24% in all cases. The results show that the MOB-ML processes can transfer information about the fundamental components of the electronic structure of water including (but not limited to) lone pairs and sigma bonds for the prediction of similar components in different molecules, including that the molecules are composed of different elements.

### Example 7: Determination of CCSD and MP2 Correlation Energies of Set of Organic Molecules

Processes in accordance with various embodiments of the invention rely upon the transferability of MOB-ML processes. Many embodiments implement MOB-ML processes across a set of organic molecules. Several embodiments include the determination of CCSD and MP2 correlation energies of sets of organic molecules from the QM7b 36 and GDB-13 37 datasets.

QM7b dataset is comprised of 7,211 plausible organic molecules with at most 7 heavy atoms. Chemical elements in QM7b can be C, H, O, N, S, and Cl. These elements are commonly used in drugs. Dataset QM7b-T is composed of molecular geometries sampled at a temperature of about 350 K. MOB-ML processes can be trained on a randomly chosen subset of QM7b-T molecules and used to predict the correlation energy of the remainder. A Δ-ML process is trained on the same molecules using kernel-ridge regression using the FCHL representation with a Gaussian kernel function (FCHL/Δ-ML), as implemented in the QML package. (See, e.g., Ramakrishnan R., J. Chem. Theory Comput., 2015, 11, 2087, Faber F. A., J. Chem. Phys., 2018, 148, 241717, the disclosure of which are herein incorporated by reference).

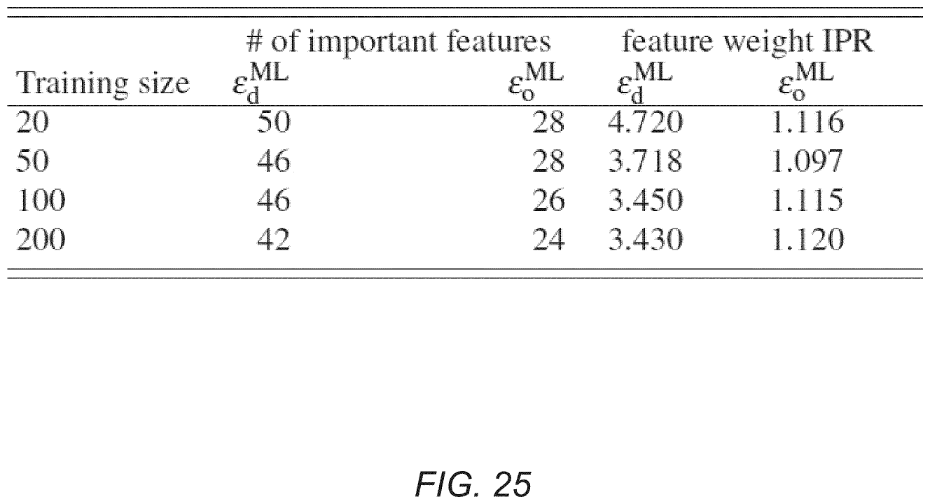

FIG. 25 presents two statistics on the feature importance as a function of the number of training molecules. One of the statistics on the feature includes the number of “important features”, which includes (but is not limited to) the permutation importance exceeding a set threshold of 2×10⁴ and 5×10⁵ for ε_{d}^{ML} and εₒ^{ML} respectively. One of the statistics on the feature includes the inverse participation ratio of the feature importance scores. Although the QM7b-T dataset contains many different chemical elements and bonding motifs, FIG. 25 reveals that the selected features can remain compact and do not grow with the number of training molecules. For a large number of training molecules, the number of selected features slightly decreases, reaching 42 and 24 selected features for ε_{d}^{ML} and εₒ^{ML} respectively in the illustrated example.

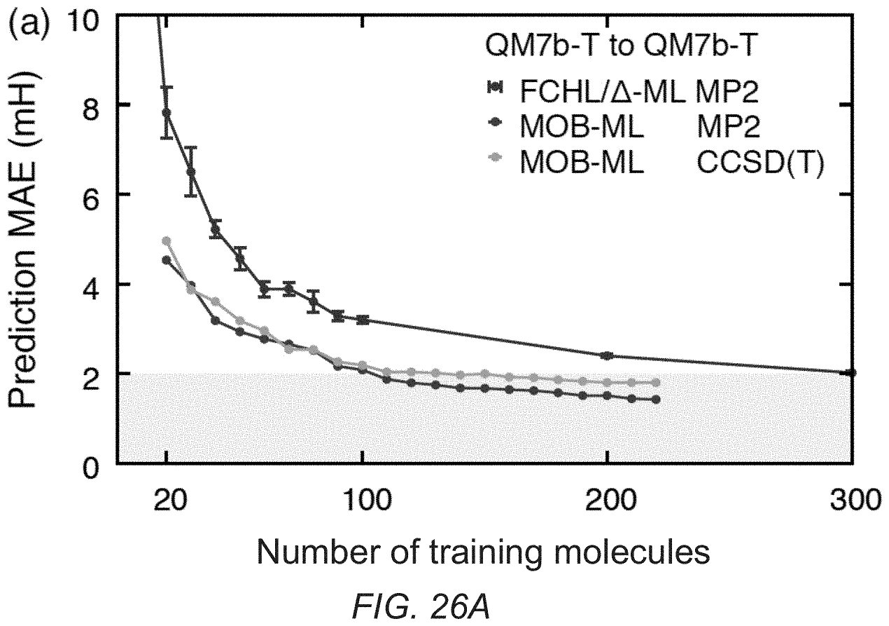

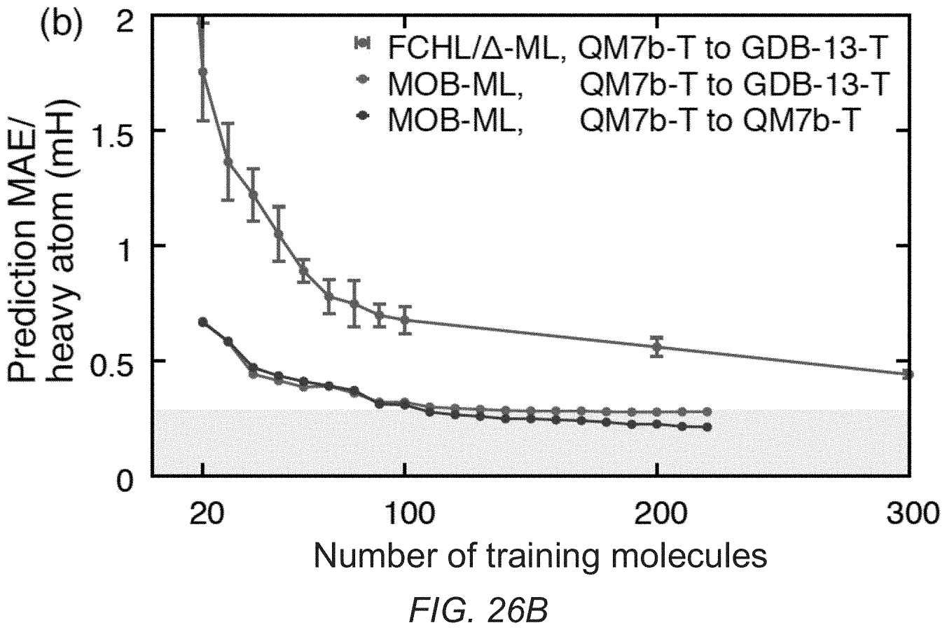

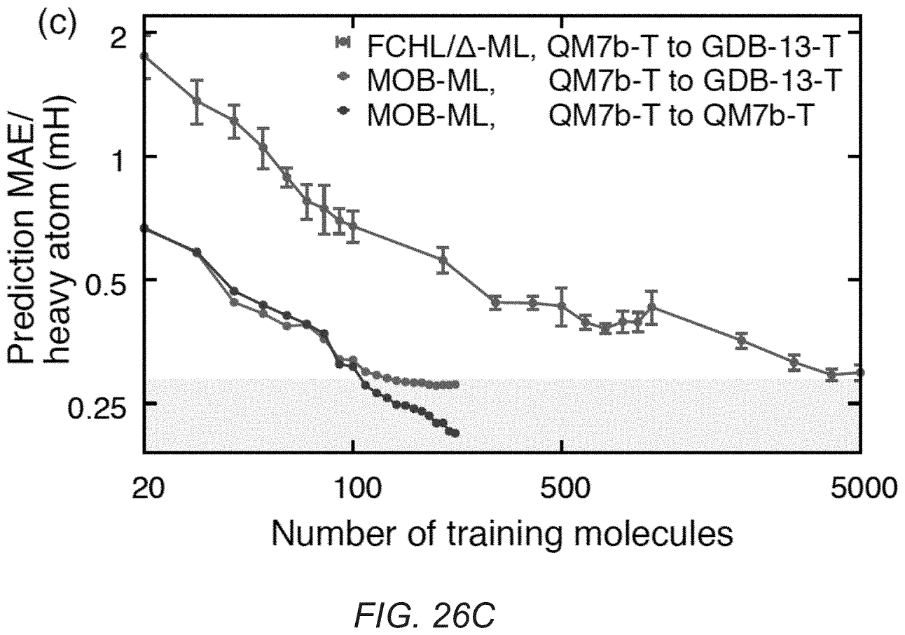

The learning curves for MOB-ML processes trained at the MP2/cc-pVTZ and CCSD(T)/cc-pVDZ levels of theory are shown in FIG. 26A, as well as the FCHL/D-ML learning curve for MP2/cc-pVTZ. At the MP2 level of theory, the MOB-ML processes achieve an accuracy of 2 mH with 110 training calculations (representing 1.5% of the molecules in the QM7b-T dataset), whereas the FCHL/Δ-ML requires over 300 training geometries to reach the same accuracy threshold in accordance with an embodiment. FIG. 26A illustrates the relative insensitivity of MOB-ML to the level of electronic structure theory also, with the learning curve for CCSD(T)/cc-pVDZ reaching 2 mH accuracy with 140 training calculations.

FIGS. 26B and 26C show that MOB-ML processes can be trained on QM7b-T molecules and used to predict results for a dataset of 13-heavy-atom organic molecules at thermalized geometries, GDB-13-T, which can include six thermally sampled geometries each of 1,000 13-heavy-atom organic molecules chosen randomly from the GDB-13 dataset. The members of GDB-13 can contain C, H, N, O, S, and Cl. The results show comparison at the MP2/cc-pVTZ level of theory. Transfer learning results as a function of the number of training molecules are shown on a linear-linear scale in FIG. 26B and on a log-log scale in FIG. 26C.

MOB-ML processes trained on 110 seven heavy-atom molecules can yield a prediction MAE of 1.89 mH for QM7b-T. Results show that a prediction MAE of 3.88 mH for GDB-13-T. Expressed in terms of size-intensive quantities, the prediction MAE per heavy atom is 0.277 mH and 0.298 mH for QM7b-T and GDB-13-T, respectively. The accuracy of the MOB-ML results are only slightly lower when the model is transferred to the dataset of larger molecules. On a per-heavy-atom basis, MOB-ML can reach chemical accuracy with the same number of QM7b-T training calculations (approximately 100), for tests on QM7b-T or GDB-13-T.

In comparison, the FCHL/Δ-ML method is significantly less transferable from QM7b-T to GDB-13-T. For models trained using 100 seven-heavy-atom molecules, the MAE per heavy atom of FCHL/Δ-ML is over twice that of MOB-ML in FIG. 26B. Moreover, MOB-ML processes can reach the per-heavy-atom chemical accuracy threshold with 140 training calculations, while the FCHL/Δ-ML processes only reach that threshold with 5000 training calculations.

### Example 8: Clustering and Classification in MOB Feature Space of Sets of Organic Molecules

Processes in accordance with various embodiments of the invention can utilize the workflow of clustering, regression, and classification for training and evaluating MOB-ML processes. Many embodiments include clustering and classification in MOB feature space. Several embodiments include locally linear clusters that overlap in sets of molecules from QM7b-T datasets using MOB-ML processes.

Many embodiments include the QM7b-T set of drug-like molecules with thermalized geometries, using the diagonal pair correlation energies ε_{d}^{ML} computed at the MP2/cc-pVTZ level. Some embodiments include randomly selection of 1000 molecules for training and perform RC on the dataset comprised of the energy labels and feature vectors, using N=20 optimized clusters. The sensitivity of RC to the choice of N can be examined.

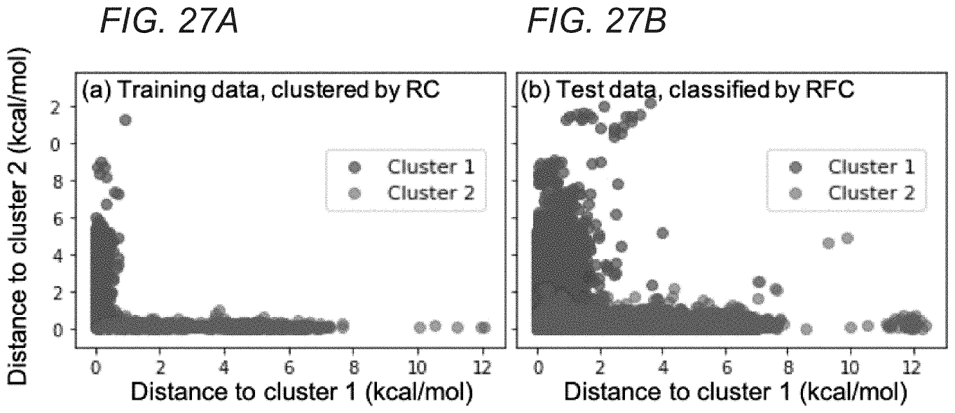

In many embodiments, the resulting clusters can be well separated, such that the datapoints for one cluster can have small distances to the cluster which it belongs to and large distances to all other clusters. In some embodiments the clusters can overlap. FIG. 27 shows the overlap for two clusters (labeled as Cluster 1 and Cluster 2) obtained from the QM7b-T diagonal-pair training data in accordance with an embodiment. FIG. 27A shows the overlap of clusters obtained via RC for the training set molecules from QM7b-T. FIG. 27B shows classification of the datapoints for the remaining test molecules from QM7b-T using RFC. Distances correspond to the linear regression metric defined in Eq. 11.

Each datapoint assigned to cluster 1 in blue color can be plotted according to its distance to both cluster 1 and cluster 2; likewise for the datapoints in cluster 2 in red color. The datapoints for which the distances to both clusters approach zero can correspond to regions of overlap between the clusters in the high dimensional space of MOB-ML features, exhibiting features similar to those described above with respect to FIG. 3.

FIG. 27B shows classification of the feature vectors into clusters. An RFC can be trained on the feature vectors and cluster labels for the diagonal pairs of 1000 QM7b-T molecules in the training set, and the classifier can be used to predict the cluster assignment for the feature vectors associated with the remaining diagonal pairs of 6211 molecules in QM7b-T. For clusters 1 and 2, the accuracy of the RFC can be analyzed by plotting the linear regression distance for each datapoint to the two clusters, as well as indicating the RFC classification of the feature vector. Each red datapoint in FIG. 27B that lies above the diagonal line of reflection is mis-classified into cluster 2. Each blue datapoint that lies below the line of reflection is mis-classified into cluster 1. FIGS. 27A and 27B illustrate that while RFC is a qualitatively correct means of classification.

### Example 9: Chemically Intuitive Clusters in Sets of Organic Molecules

Processes in accordance with various embodiments of the invention can utilize chemically intuitive clusters during regression clustering of MOB-ML processes. Many embodiments include evaluating consistency of the clustering and classification processes with chemical intuition.

Many embodiments include a training set of 500 randomly selected molecules from QM7b-T and regression clustering for the diagonal pair correlation energies Er′ with a range of total cluster numbers up to N=20. For each clustering, an RFC can be trained. Each trained RFC can be independently applied to a set of test molecules with easily characterized valence molecular orbitals to see how the feature vectors associated with valence occupied LMOs can be classified among the optimized clusters.

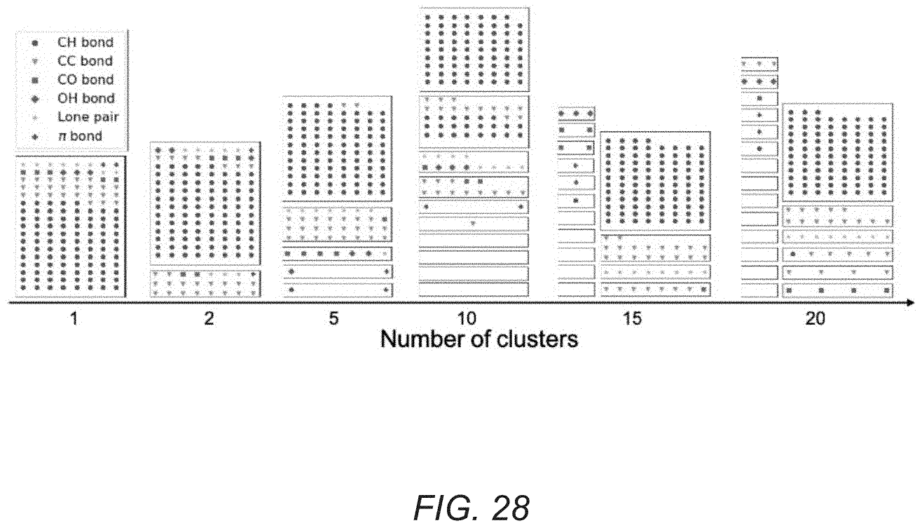

FIG. 28 shows results of clustering and classification in terms of chemical intuition. The trained classifier can be applied to a set of test molecules including (but are not limited to) CH₄, C₂H₆, C₂H₄, C₃H₈, CH₃CH₂OH, CH₃OCH₃, CH₃CH₂CH₂CH₃, CH₃CH(CH₃)CH₃, CH₃CH₂CH₂CH₂CH₂CH₂CH₃, (CH₃)₃CCH₂OH, and CH₃CH₂CH₂CH₂CH₂CH₂OH, which have chemically intuitive LMO types. As can readily be appreciated, the specific features used as LMO type test molecules are largely only limited to the requirements of specific applications. The LMOs can be resolved according to type by the classifier as the number of clusters increases. Empty boxes correspond to clusters into which none of the LMOs from the test set can be classified.

FIG. 28 shows the agreement between chemical intuition and the predictions of the RFC in accordance with an embodiment. As the number of clusters increases, the feature vectors associated with different valence LMO types can be resolved into different clusters. With a sufficiently large number of clusters (15 or 20), each cluster can be dominated by a single type of LMO while each LMO type can be assigned to a small number of different clusters. The empty boxes in FIG. 28 reflect that the training set can contain a larger diversity of LMO types than the 11 test molecules. The observed consistency of the clustering/classification method with chemical intuition can be promising for the accurate local regression of pair correlation energies in many embodiments. Clustering and classification of chemical systems in MOB-ML feature space can provide a powerful and highly general way of mapping the structure of chemical space for other applications, including explorative and active ML applications.

### Example 10: Sensitivity to the Number of Clusters in Clustering/Regression/Classification Processes

Processes in accordance with various embodiments of the invention can utilize the sensitivity of clustering, regression, and classification workflow MOB-ML processes. Many embodiments include sensitivity of clustering, regression, and classification processes for the diagonal and off-diagonal contributions to the correlation energy for the QM7b-T set of molecules.

Many embodiments include the mean absolute error (MAE) of the MOB-ML predictions for the diagonal (Σᵢεᵢᵢ) and off-diagonal (Σ_{i≠}εᵢᵢ) contributions to the total correlation energy, as a function of the number of clusters, N, used in the regression clustering. Several embodiments include the MOB-ML processes employ linear regression and RFC classification (i.e., the RC/LR/RFC protocol). The training set can be comprised of 1000 randomly chosen molecules from QM7b-T, and the test set can contain the remaining molecules in QM7b-T.

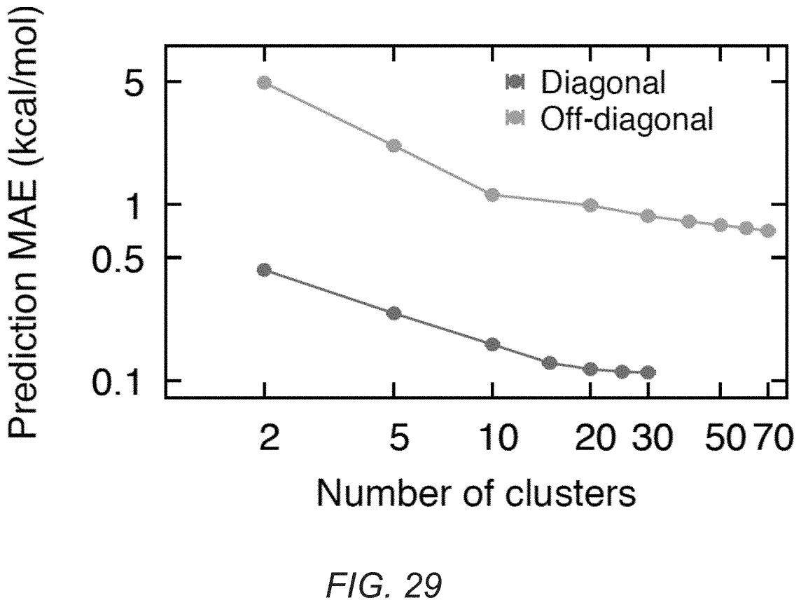

FIG. 29 show the sensitivity of MOB-ML predictions for the diagonal and off-diagonal contributions to the correlation energy for the QM7b-T set of molecules, using a subset of 1000 molecules for training and the RC/LR/RFC protocol. The standard error of the mean (SEM) for the predictions can be smaller than the size of the plotted points. The prediction accuracy for both the diagonal and off-diagonal contributions can improve with N. For the diagonal contributions, the accuracy can improve most rapidly up to approximately 20 clusters, exhibiting features similar to those described above with respect to FIG. 29. For the off-diagonal contributions, a larger number of clusters can be useful for reducing the MAE, as the greater variety of feature vectors can be created from pairs of LMOs rather than only individual LMOs.

### Example 11: Sensitivity to Performance and Training Costs of MOB-ML Processes with Regression Clustering

Processes in accordance with various embodiments of the invention rely upon the sensitivity of number of employed clusters of clustering, regression, and classification processes. Many embodiments include learning curves of MOB-ML processes applied to MP2/cc-pVTZ and CCSD(T)/cc-pVDZ correlation energies for the QM7b-T set of molecules.

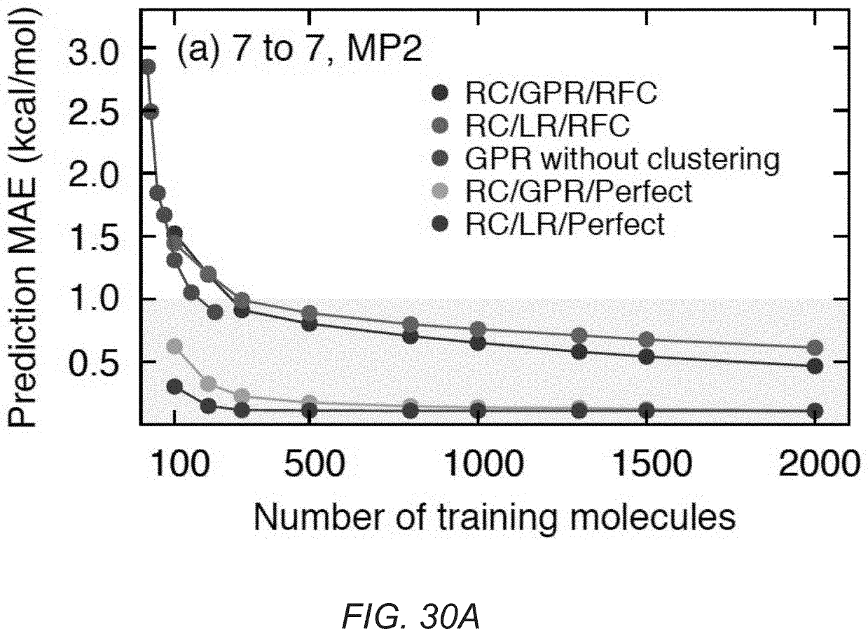

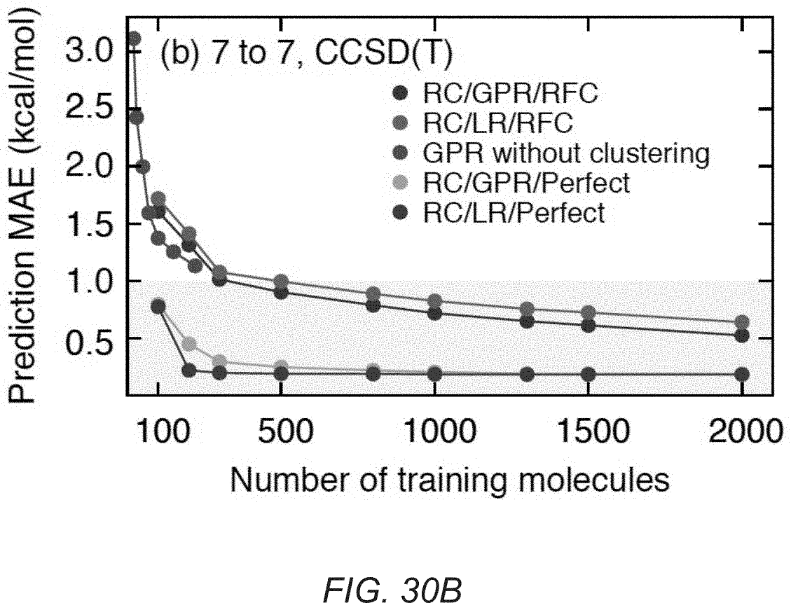

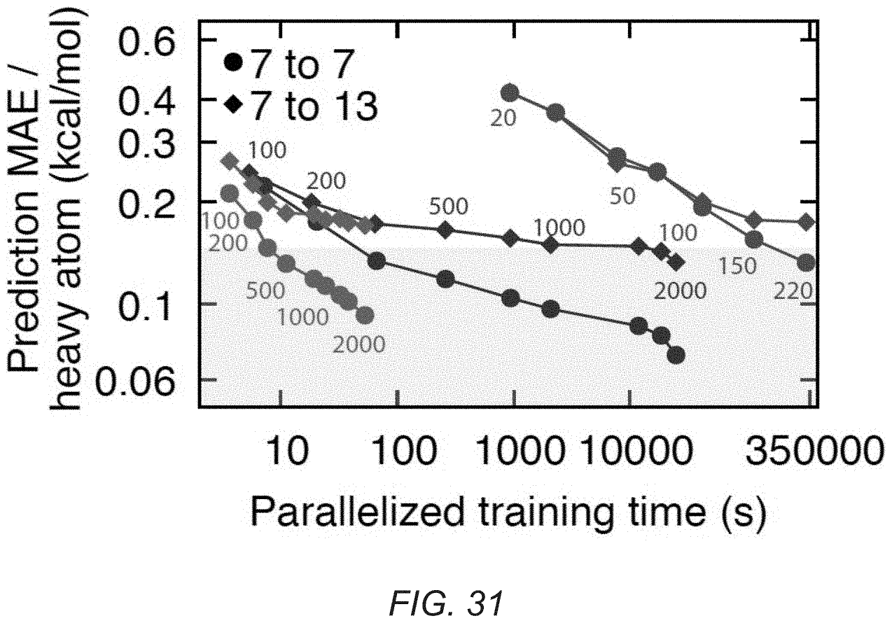

Many embodiments include the effect of clustering on the accuracy and training costs of MOB-ML for applications to sets of drug-like molecules with up to seven heavy atoms. FIG. 30A shows learning curves (on a linear-linear scale) for various embodiments of MOB-ML processes applied to MP2/cc-pVTZ correlation energies, with the training and test sets corresponding to non-overlapping subsets of QM7b-T. The gray shaded area corresponds to a MAE of 1 kcal/mol per seven heavy atoms. The prediction SEM is smaller than the plotted points. FIG. 30A shows that the use of RC with RFC (i.e., RC/GRP/RFC and RC/LR/RFC) can lead to slightly less efficient learning curves than implementation without clustering, at least when efficiency is measured in terms of the number of training molecules. Both the RC/GPR/RFC and RC/LR/RFC protocols require approximately 300 training molecules to reach the 1 kcal/mol per seven heavy atoms threshold for chemical accuracy, whereas MOB-ML without clustering requires approximately half as many training molecules. The classifier can be the dominant source of prediction error in the results. Comparison of results using RFC versus the perfect classifier can reveal a dramatic reduction in the prediction error, regardless of the regression method. There is potentially much to be gained from the development of improved classifiers for MOB-ML applications. With a perfect classifier, the LR can slightly outperform GPR, given that the clusters can be optimized to be locally linear. GPR can slightly outperform LR in combination with the RFC, indicating that GPR is less sensitive to classification error that LR.

FIG. 30B shows learning curves (on a linear-linear scale) for various embodiments of MOB-ML processes applied to CCSD(T)/cc-pVDZ correlation energies, with the training and test sets corresponding to non-overlapping subsets of QM7b-T. The same trends emerge as the ones at the MP2/cc-pVTZ level of theory. The training efficiency of MOB-ML with respect to the size of the reference dataset can be found to be largely insensitive to the level of electronic structure theory.

Several embodiments include the training costs and transferability of MOB-ML models that employ RC. In FIG. 31, the MOB-ML is trained on random subsets of molecules from QM7b-T with up to seven heavy atoms, and predictions are made either on the remaining molecules of QM7b-T (circles) or on the GDB-13-T set (diamonds). MOB-ML can substantially outperform the FCHL atom-based-feature method in terms of transferability from small to large molecules, similar to those results described above with respect to FIG. 23. The parallelization of the training steps can be implemented as follows. Within the RC step, the LR for each cluster can be performed independently on a different core of a 16-core Intel Skylake (2.1 GHz) CPU processor. Within the regression step, the LR or GPR for each cluster can be likewise performed independently on a different core. For RFC training, 200 parallel cores can be applied using the parallel implementation of SCIKIT-LEARN, since there are 200 trees. The regression and RFC training can be independent of each other and thus can also be trivially parallelizable.

For the predictions for seven-heavy-atom molecules (circles), FIG. 31 shows that RC can lead to large improvements in the efficiency of the MOB-ML wall-clock training costs. Although it requires somewhat more training molecules than MOB-ML without clustering, MOB-ML with clustering can enable chemical accuracy to be reached with the training cost reduced by a factor of approximately 4500 for RC/GPR/RFC and of 35000 for RC/LR/RFC. For predictions within the QM7b-T set, chemical accuracy can be achieved using RC/LR/RFC with a wall-clock training time of only 7.7 s.

FIG. 31 demonstrates the transferability of the MOB-ML processes for predictions on the GDB-13-T set of thirteen-heavy-atom molecules (diamonds). The degradation in the MAE per atom can be greater for the RC/LR/RFC than for RC/GPR/RFC. However, the RC/GPR/RFC can enable predictions on GDB-13-T (blue, diamonds) that meet the per-atom threshold of chemical accuracy. The threshold cannot be achievable without clustering (green, diamonds) due to the prohibitive training costs involved.

The improved efficiency of MOB-ML training with the use of clustering can arise from the cubic scaling of standard GPR in terms of training time (O(M³), where M is number of training pairs). Trivial parallelization over the independent regression of the clusters can reduce training time cost to the cube of the size of the largest cluster. Other kernel-based ML methods with high complexity in training time, like Kernel Ridge Regression, can similarly benefit from clustering. GPR regression can dominate the total training (and prediction) costs for the RC/GPR/RFC implementation, whereas training the RFC can dominate the training costs for RC/LR/RFC. In addition to improved efficiency in terms of training time, clustering can also bring benefits in terms of the memory costs for MOB-ML training, due to the quadratic scaling of GPR memory costs in terms of the size of the dataset.

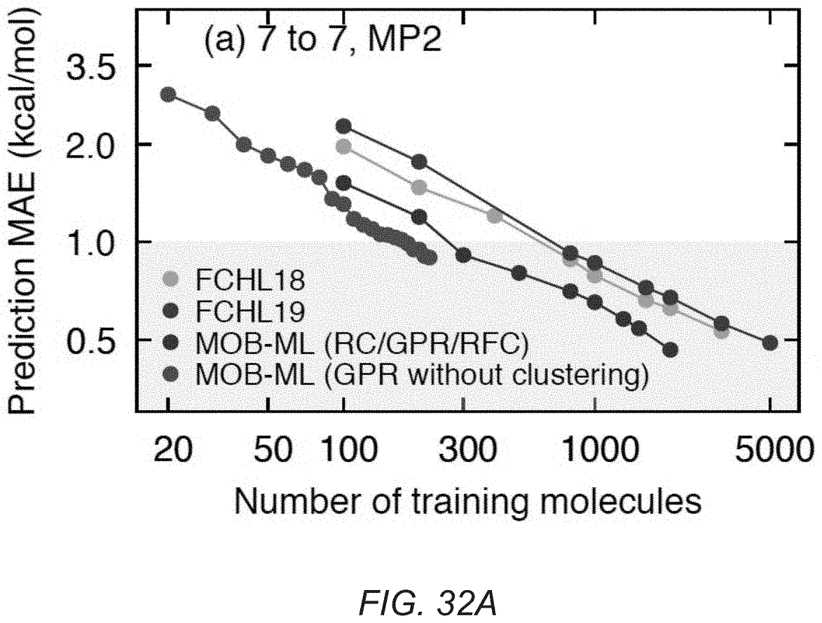

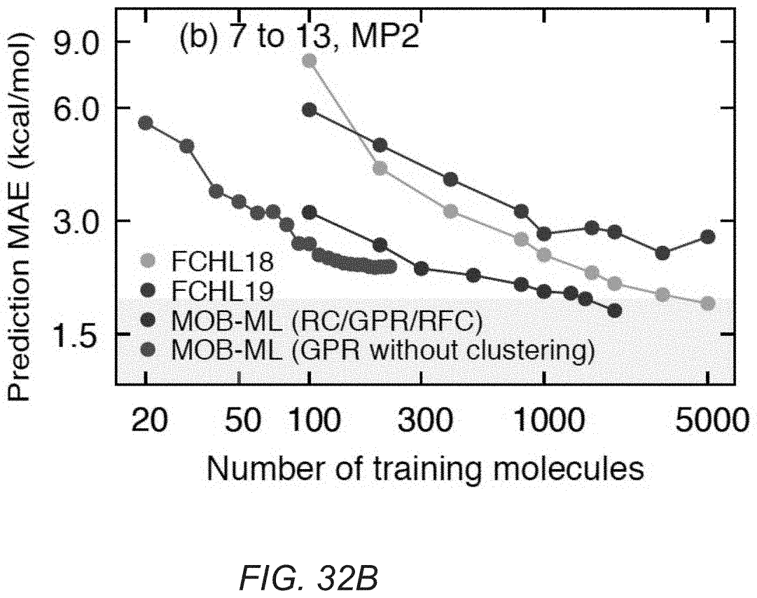

For the learning curves, some embodiments compare the results for MOB-ML both with and without clustering to Faber-Christensen-Huang-Lilienfeld (FCHL) features. FIG. 32 shows the various learning curves for the MP2/cc-pVTZ correlation energies. For FIG. 32A, the training and test sets correspond to non-overlapping subsets of QM7b-T, and FIG. 32B shows the transferability of the same models trained using QM7b-T to predict the energies for GDB-13-T. FIG. 32A shows that MOB-ML RC/GPR/RFC can require slightly more training geometries than MOB-ML without clustering. Yet both MOB-ML protocols can be more efficient in terms of training data than either the FCHL18 or FCHL19 implementations.

### Example 12: Capping the Cluster Size of Regression Clustering

Processes in accordance with various embodiments of the invention can utilize the sizes of clusters of clustering, regression, and classification processes. Many embodiments include effect of cluster-size capping on the prediction accuracy and training costs for MOB-ML with RC.

Many embodiments include that capping the number of datapoints in the largest cluster can achieve additional computational savings and adequate prediction accuracy. Some embodiments include Sₘₐₓ^{N}^{_{cap}} as the number of datapoints in the largest cluster obtained when the RC with the greedy algorithm is applied to a training dataset of N_{cap} molecules from QM7b-T. Upon specifying N_{cap} (and thus Sₘₐₓ^{N}^{_{cap}}), the RC/GPR/RFC implementation can be modified. For a given number of training molecules (which will typically exceed N_{cap}), the RC step can be performed as normal. However, at the end of the RC step, datapoints for clusters whose size exceeds Sₘₐₓ^{N}^{_{cap}} can be discarded at random until all clusters contain Sₘₐₓ^{N}^{_{cap}} or fewer datapoints. The GPR and RFC training steps can be performed as before, except using this set of clusters that are capped in size. The precise value of Sₘₐₓ^{N}^{_{cap}} can vary slightly depending on which training molecules are randomly selected for training and the convergence of the greedy algorithm, but typical values for Sₘₐₓ^{N}^{_{cap}} are 672, 1218, 1863, 3005, and 4896 for N_{cap}=100, 200, 300, 500 and 800, respectively, and those values are used for the numerical tests.

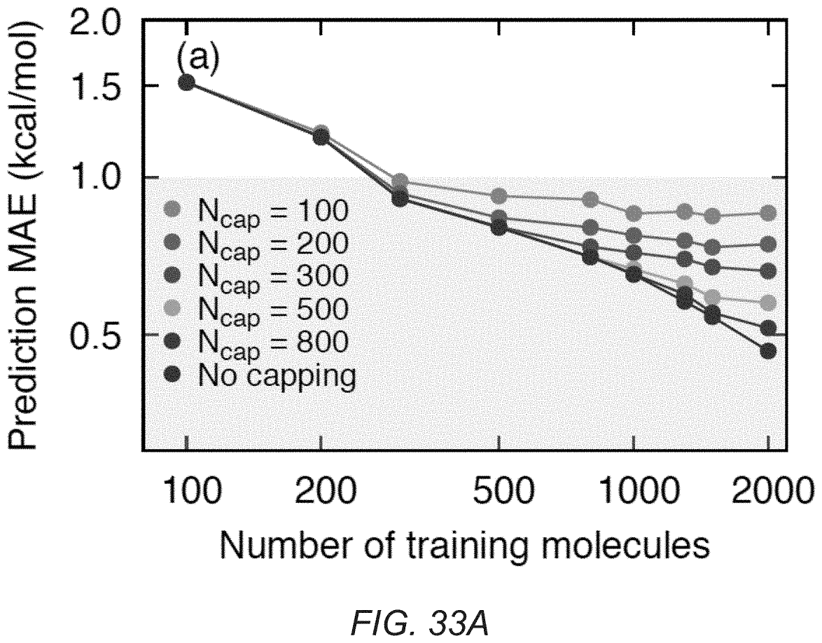

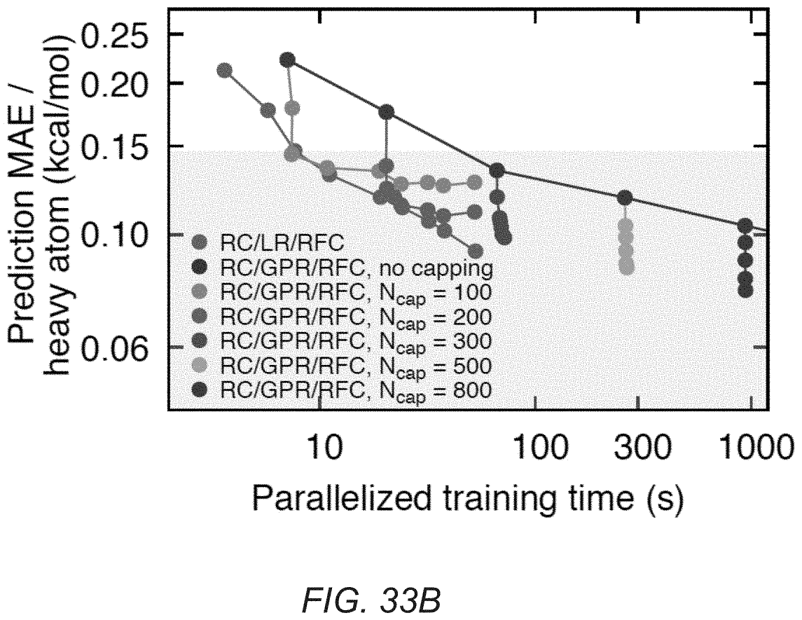

FIG. 33 shows the effect of cluster-size capping on the prediction accuracy and training costs for MOB-ML with RC. Results reported for correlation energies at the MP2/cc-pVTZ level, with the training and test sets corresponding to non-overlapping subsets of the QM7b-T set of druglike molecules with up to heavy seven atoms. FIG. 33A plots MOB-ML prediction MAE versus the number of training molecules, with the clusters capped at various maximum sizes. The RC/GPR/RFC curve without capping is reproduced from FIG. 30A. The gray shaded area corresponds to a MAE of 1 kcal/mol. FIG. 33B plots MOB-ML prediction MAE per heavy atom versus parallelized training time as a function of the number of training molecules, as in FIG. 31. The results for MOB-ML with clustering and without capping cluster size (RC/LR/RFC, red; RC/GPR/RFC, blue) are reproduced from FIG. 31. Also, the results for RC/GPR/RFC with various capping sizes N_{cap} are shown. the gray shaded area corresponds to 1 kcal/mol per seven heavy atoms. The prediction SEM is smaller than the plotted points.

FIG. 33A demonstrates that capping the maximum cluster size can allow for substantial improvements in accuracy when the number of training molecules exceeds N_{cap}. Specifically, the figure shows the effect of capping on RC/GPR/RFC learning curves for MP2/cc-pVTZ correlation energies, with the training and test sets corresponding to non-overlapping subsets of QM7b-T. As a baseline, with 100 training molecules, the RC/GPR/RFC implementation can yield a prediction MAE of approximately 1.5 kcal/mol. However, if the maximum cluster size is capped at N_{cap}=100 and 300 training molecules are employed, then the prediction MAE drops to approximately 1.0 kcal/mol while the parallelized training cost for RC/GPR/RFC is unchanged so long as it can remain dominated by the size of the largest cluster. FIG. 33A shows that the learning curves can saturate at higher prediction MAE values when smaller values of N_{cap} are employed. Nonetheless, it demonstrates that if additional training data is available, then the prediction accuracy for MOB-ML with RC can be substantially improved while capping the size of the largest cluster.

FIG. 33B demonstrates the actual effect of capping on the parallelized training time, plotting the prediction MAE versus parallelized training time as a function of the number of training molecules. For reference, the results obtained using RC/LR/RFC and RC/GPR/RFC without capping are reproduced from FIG. 31. The RC/GPR/RFC results obtained with capping exactly overlap those obtained without capping when the number of training molecules is not greater than N_{cap}. However, for each value of N_{cap}, a sharp drop in the prediction MAE can be seen when the number of training molecules begins to exceed N_{cap}, demonstrating that prediction accuracy can be greatly improved with minimal increase in parallelized training time. For example, it is seen that for RC/GPR/RFC with N_{cap}=100, chemical accuracy can be reached with only 7.4 s of parallelized training, slightly less than even RC/LR/RFC. For small values of N_{cap}, the prediction MAE can eventually level-off versus the training time, since the RFC training step becomes the dominant contribution to the training time.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A method of synthesizing a molecule comprising,
obtaining a set of molecular orbitals for a molecular system using a computer system;
generating a set of molecular-orbital-based features based upon the set of molecular orbitals of the molecular system using the computer system;
determining at least one molecular system property based on the set of features using a molecular-orbital-based machine learning (MOB-ML) model implemented on the computer system; and
when the determined at least one molecular system property satisfies at least one criterion by the computer system, synthesizing the molecular system.

2. The method of claim 1, wherein the set of molecular-orbital-based features comprises an attributed graph representation of molecular-orbital-based features.

3. The method of claim 1, wherein:
the molecular system is one of a plurality of candidate molecular systems; and
determining when the determined at least one molecular system property satisfies at least one criterion further comprises:
generating a set of molecular-orbital-based features based upon sets of molecular orbitals for each of the candidate molecular systems;
determining at least one molecular system property for each of the candidate molecular systems based on the set of molecular-orbital-based features of each of the candidate molecular systems using the MOB-ML model;
screening the candidate molecular systems based upon the at least one molecular system property determined for each of the candidate molecular systems; and
identifying the molecular system based upon the screening.

4. The method of claim 1, further comprising training the MOB-ML model to learn relationships between sets of molecular-orbital-based features and molecular system properties using a training dataset describing a plurality of molecular systems and their molecular system properties.

5. The method of claim 4, wherein training the MOB-ML model to learn relationships between sets of molecular-orbital-based features and molecular system properties further comprises:
obtaining a set of molecular orbitals for each molecular system in the training dataset of molecular systems by determining occupied molecular orbitals; and
obtaining a set of molecular-orbital-based features based upon at least the occupied molecular orbitals.

6. The method of claim 5, wherein a localization process is used to determine occupied molecular orbitals.

7. The method of claim 5, wherein obtaining the set of molecular-orbital-based features further comprises performing a dimensionality reduction process on an initial set of features.

8. The method of claim 7, wherein the dimensionality reduction process is selected from the group consisting of selecting the molecular-orbital-based features from the initial set of features, and applying a transformation process to the initial set of features to obtain the molecular-orbital-based features.

9. The method of claim 8, wherein the transformation process is selected from the group consisting of subspace embedding and autoencoding.

10. The method of claim 4, wherein training the MOB-ML model comprises at least one process selected from the group consisting of regression clustering, regression, and classification.

11. The method of claim 10, wherein training the MOB-ML model comprises at least regression process selected from the group consisting of Gaussian Process Regression, Neural Network Regression, Linear Regression, and Kernel Ridge Regression with feature selection based on Random Forest Regression, Kernel Ridge Regression without feature selection based on Random Forest Regression, and Kernel Ridge Regression with feature transformation based on Principle Component Analysis.

12. The method of claim 1, wherein the molecular system comprises at least one of atoms, molecular bonds, and molecules formed by atoms and molecular bonds.

13. The method of claim 1, wherein the set of features includes molecular-orbital-based (MOB) features comprising an energy operator.

14. The method of claim 13, wherein the molecular-orbital-based features further comprise at least one feature selected from the group consisting of:
elements from a Fock matrix,
elements from a Coulomb matrix, and
elements from an exchange matrix.

15. The method of claim 1, wherein the at least one molecular system property comprises at least one property selected from the group consisting of quantum correlation energy, force, vibrational frequency, dipole moment, response property, excited state energy and force, and spectrum.

16. The method of claim 1, wherein the synthesized molecular system comprises at least one molecule selected from the group consisting of a catalyst, an enzyme, a pharmaceutical, a protein, an antibody, a surface coating, a nanomaterial, a semiconductor, a solvent for a battery, and an electrolyte for a battery.

17. A method of screening a set of candidate molecular systems comprising:
obtaining set of molecular orbitals fora plurality of candidate molecular systems using a computer system;
generating a set of molecular-orbital-based features for each candidate molecular system based upon sets of molecular orbitals for each of the candidate molecular systems using the computer system;
determining at least one molecular system property for each of the candidate molecular systems based on the set of molecular-orbital-based features of each of the candidate molecular systems using a molecular-orbital-based machine learning (MOB-ML) model implemented on the computer system;
screening the candidate molecular systems to identify at least one molecular system possessing at least one molecular system property that satisfies at least one criterion based upon the at least one molecular system property determined for each of the candidate molecular systems using the computer system; and
generating a report describing the at least one molecular system identified during the screening of the candidate molecular systems using the computer system.

18. A method of synthesizing a molecular system using an inverse molecule design process comprising:
searching for a set of molecular-orbital-based features having at least one molecular system property predicted by a molecular-orbital-based machine learning (MOB-ML) model that satisfies at least one criterion using a computer system, where the MOB-ML model is trained to receive a set of features of a molecular system and output an estimate of at least one molecular system property;
mapping a located set of molecular-orbital-based features to an identified molecular system using a feature-to-structure map using the computer system, where the feature-to-structure map is trained to map a set of molecular-orbital-based features to a corresponding molecule structure;
screening the identified molecular system based upon at least one screening criterion using the computer system; and
when the identified molecular system satisfies the at least one screening criterion, synthesizing the identified molecular system.

19. The method of claim 18, wherein searching for a set of molecular-orbital-based features having at least one molecular system property predicted by the MOB-ML model that satisfies at least one criterion further comprises using at least one generative model to generate candidate sets of features.

20. The method of claim 19, wherein the generative model is selected from the group consisting of a variational autoencoder (VAE) and a Generative Adversarial Network (GAN).

21. A method of training a molecular-orbital-based machine learning (MOB-ML) model to predict at least one molecular system property from a set of molecular orbitals for a molecular system comprising:
obtaining a training dataset of molecular systems and their molecular system properties using a computer system;
generating a set of molecular-orbital-based features for each molecular system in the training dataset based upon a set of molecular orbitals for each of the candidate molecular systems using the computer system;
training a ML model to learn relationships between the set of molecular-orbital-based features of each molecular system in the training dataset and the molecular system properties of each of the molecular systems in the training dataset using the computer system; and
utilizing the MOB-ML model to predict at least one molecular system property for a specific molecular system based upon a set of molecular-orbital-based features generated for the specific molecular system based upon a set of molecular orbitals for the specific molecular system.

22. The method of claim 21, wherein obtaining a training dataset of molecular systems and their molecular system properties further comprises:
generating a set of molecular-orbital-based features for the specific molecular system based upon a set of molecular orbitals for the specific molecular system using the computer system;
retrieving molecular-orbital-based features from a database based upon proximity between a retrieved molecular-orbital-based feature and a molecular-orbital-based feature from the set of molecular-orbital-based features for the specific molecular system; and
forming the training dataset using the retrieved molecular systems.

23. The method of claim 21, wherein training the MOB-ML model to learn relationships between the sets of molecular-orbital-based features of each molecular system in the training dataset and the molecular system properties of each of the molecular systems in the training dataset further comprises utilizing a transfer learning process to train an MOB-ML model previously trained to determine the relationship between a molecular-orbital-based features of a molecular system and a different set of molecular system properties.

24. The method of claim 21, wherein training the MOB-ML model to learn relationships between the sets of molecular-orbital-based features of each molecular system in the training dataset and the molecular system properties of each of the molecular systems in the training dataset further comprises utilizing an online learning process to update a previously trained MOB-ML model.
